SEQUENCE LISTING (8 sequences)

SEQ ID NO: 1 - DNA, 30 bp, Artificial
  Sense primer Template-switch oligo Universal. The 3 last G are RNA bases and not DNA bases, contrary to other bases, all DNA.
aagcagtggtatcaacgcagagtacatggg

SEQ ID NO: 2 - DNA, 22 bp, Artificial
  Antisense primer kappa mIGK chain for RT
ttgtcgttcactgccatcaatc

SEQ ID NO: 3 - DNA, 21 bp, Artificial
  Antisense primer lambda mIGK chain for RT
ggggtaccatctaccttccag

SEQ ID NO: 4 - DNA, 20 bp, Artificial
  Antisense primer mIGHG heavy chain for RT
agctgggaaggtgtgcacac

SEQ ID NO: 5 - DNA, 22 bp, Artificial
  Sense primer ISPCR Universal
aagcagtggtatcaacgcagag

SEQ ID NO: 6 - DNA, 24 bp, Artificial
  Antisense primer kappa mIGK chain for PCR
acattgatgtctttggggtagaag

SEQ ID NO: 7 - DNA, 20 bp, Artificial
  Antisense primer lambda mIGK chain for PCR
atcgtacacaccagtgtggc

SEQ ID NO: 8 - DNA, 20 bp, Artificial
  Antisense primer mIGHG heavy chain for PCR
gggatccagagttccaggtc

(19) United States
(12) Patent Application Publication
(10) Pub. No.: US 2024/0182550 A1
(43) Pub. Date: Jun. 6, 2024

(54) ANTIBODIES, FRAGMENTS OR DERIVATIVES SPECIFICALLY BINDING TO A PROTEIN ANTIGEN CAPABLE OF BINDING TO NUCLEIC ACIDS AND USES OF SAME

(71) Applicant: COMMISSARIAT A L'ENERGIE ATOMIQUE ET AUX ENERGIES ALTERNATIVES, Paris (FR)

(72) Inventors: Michel LEONETTI, Gif-sur-Yvette Cedex (FR); Oscar PEREIRA RAMOS, Gif-sur-Yvette Cedex (FR); Stephanie SIMON, Gif-sur-Yvette Cedex (FR); Gwenaelle LE ROUX, Gif-sur-Yvette Cedex (FR); Nathalie MOREL, Gif-sur-Yvette Cedex (FR)

(21) Appl. No.: 18/547,471

(22) PCT Filed: Feb. 24, 2022
(86) PCT No.: PCT/FR2022/050338

§ 371 Date: Aug. 22, 2023

(30) Foreign Application Priority Data
Feb. 24, 2021 (FR) FR2101789

(51) Int. Cl. C07K 16/10; A61K 39/00; A61P 37/06; C07K 16/08; C07K 16/18

(52) U.S. Cl. CPC C07K 16/1003; A61P 37/06; C07K 16/082; C07K 16/084; C07K 16/1018; C07K 16/1045; C07K 16/1072; C07K 16/18; A61K 39/00

(57) ABSTRACT

The present invention relates to a specific antibody of a protein antigen capable of binding to nucleic acids, or a fragment or a derivative of such an antibody binding to the antigen, for use as a drug, in particular in the treatment or prevention of inflammation, due especially to an infection or an autoimmune disease, characterised in that the antibody, fragment or derivative has a reduced capacity to bind to the FcγRIIA receptor and/or an increased capacity to bind to the FcγRIIB receptor. The antibody, fragment or derivative is preferably without an Fc domain, or with a modified Fc domain with a reduced capacity to bind to FcγRIIA and optionally FcγRIIA (or even a reduced capacity to bind to all FcγR), and/or with a modified Fc domain with an increased capacity to bind to FcγRIIB.

### REFERENCE TO SEQUENCE LISTING SUBMITTED ELECTRONICALLY

The instant application contains a Sequence Listing which has been submitted electronically in ASCII format and is hereby incorporated by reference in its entirety. Said ASCII copy, created on Dec. 6, 2023, is named B383347US_Sequence listing.txt and is 2,100 bytes in size.

### FIELD OF THE INVENTION

The present invention is in the field of drug therapies for resolving inflammatory disorders induced by immune complexes. It concerns an antibody specific for a protein antigen capable of binding to nucleic acids, or to an antigen-binding fragment or derivative of such an antibody, for its use as a medicament, in particular in the treatment or prevention of inflammation, especially that due to an infection or an autoimmune disease, characterized in that the antibody, fragment or derivative has a reduced capacity for binding to the FcγRIIA receptor and/or an increased capacity of binding to the FcγRIIB receptor. The antibody, fragment or derivative preferably has no Fc domain, or has a modified Fc domain with reduced capacity to bind to FcγRIIA and possibly FcγRIIIA (or with reduced capacity to bind to all FcγRs), and/or with a modified Fc domain with increased FcγRIIB binding capacity.

### PRIOR ART

Acute and uncontrolled inflammatory reactions can be triggered during infection by microorganisms and contribute to pathogenesis (Cecconi et al., 2018; Horvath et al., 2011). These deleterious reactions can occur early, at the onset of infection (Hawkins et al., 2018; Tisoncik et al., 2012). However, they can also be triggered later, when the specific immune response occurs (Hung, 2020). In this second case, it has been observed that immune complexes (IC), resulting from the association of subunits of the pathogen with specific antibodies (Ab), induce a hyperproduction of cytokines, especially inflammatory, which tend to increase pathology. This phenomenon has especially been observed in the case of dengue virus infections (OhAinle et al., 2011). It is postulated that it may occur in SARS-COV-2 infections and may contribute to increased disease severity (Fu et al., 2020; Tillett et al., 2021; Torres et al., 2020).

Chronic inflammatory reactions can also be triggered during autoimmune disorders and can be disabling in the patient. These reactions can especially be caused by the presence of self antigen (SA) specific antibodies (Sospedra & Martin, 2016). In this case, the presence of an IC resulting from the association of self antigens with antibodies (Ab) could be responsible for inflammation.

Thus, ICs could induce inflammation responsible for pathological effects in certain infections by microorganisms as well as in certain inflammatory and/or autoimmune diseases (Kapingidza et al., 2020). The implementation of therapeutic approaches to counteract these inflammatory effects is therefore a crucial issue to improve the clinical condition of patients.

An important part of anti-inflammatory treatments is based on medicaments capable of blocking cytokines involved in inflammation or associated receptors (Dinarello, 2010). However, these medicaments tend to block all activities mediated by the cytokine or receptor considered. Yet, these latter molecules can also play crucial roles in many other physiological processes necessary to maintain homeostasis. As a result, these anti-inflammatory approaches can induce undesirable side effects and thus be less well tolerated by the patient.

Another approach to block inflammation is intravenous immunoglobulin (IVIG) injection. IVIGs are non-specific for the pathology. It is postulated that, following their injection, they will interact with the Fc gamma IIb receptor (FcγRIIb). FcγRIIb will therefore transmit an inhibition signal and thus allow a blockade of the inflammatory reaction mediated by ICs (Ben Mkaddem et al., 2019). This relatively effective approach, however, tends to abrogate the activity of all FcγRIIbs in the body. Treatment could thus disrupt other physiological processes in which FcγRIIbs are involved in order to maintain homeostasis. This type of treatment may therefore be less well tolerated by the patient.

Another anti-inflammatory approach consists of using antibodies capable of blocking the activator Fc gamma receptors, such as FcγRIIA and FcγRIIIA, in order to limit the inflammatory process mediated by ICs (Ben Mkaddem et al., 2019). This approach will lead to the blocking of all the FcγRIIA and FcγRIIIA of the body and could thus disturb other physiological processes in which these receptors would be involved in order to maintain homeostasis. This type of treatment, like the approaches described above, may be less well tolerated by the patient.

Existing approaches to reduce IC-induced inflammation in some microorganism infections as well as inflammatory and/or autoimmune diseases are therefore not specific enough, and there is therefore a need for new, more effective approaches, with less risk of side effects and that preserve the ability of the body to fight against other pathologies.

### SUMMARY OF THE INVENTION

There are many proteins derived from microorganisms or humans that have the ability to bind nucleic acids (hereinafter ‘protein antigens with the ability to bind nucleic acids’ or ‘Ag_{nuc}’). Moreover, it has been shown that antibody responses are generated against such proteins, especially against viral nucleocapsid proteins and human ribonucleoproteins (Hoffman & Greidinger, 2000; Leung et al., 2004; Migliorini et al., 2005). The presence of ICs containing an Ag_{nuc} (‘IC_{nuc}’ hereinafter) is known in humans and has in some cases been associated with the pathogenesis of the disease (Cafaro et al., 2019 Carter et al., 2000; Choi et al., 2020; Fenouillet et al., 1993; Ghiggeri et al., 2019; Hashida et al., 1997; Hu, 2002; Kamar et al., 2017; Ni et al., 2020; Pisetsky & Lipsky, 2020; Rumbaugh et al., 2013; Schulte-Pelkum et al., 2009; To & Petri, 2005).

Recent work on coronaviruses and the role of ICs in the onset of severe forms suggested, however, that IC_{nuc} could play a protective role and would not encourage blocking their activating activity. Indeed, the team of Gao et al. (Gao et al., 2020) showed that three Ag_{nuc}—corresponding, respectively, to the nucleocapsid proteins of SARS-COV, MERS-COV and SARS-COV-2 coronaviruses—can each trigger pathological inflammatory effects and that injection, in the previously infected animal, of an integral and specific Ab for the Ag_{nuc} of SARS-COV-2 may block these deleterious effects. In addition, Zohar et al. (Zohar et al., 2020) have shown that people who do not survive the disease have antibodies that have a reduced ability to bind Fcγ receptors. Finally, Combes et al. (Combes et al., 2021) has shown that patients with severe forms develop antibodies that engage the inhibitor FcγIIB receptor.

In the context of the present invention, the inventors have been interested in Ag_{nuc} and have shown, surprisingly, that IC_{nuc} (especially those of the nucleocapsid protein of the SARS-COV-2 virus) can trigger inflammatory responses. They then observed that IC_{nuc} formed solely with Ab fragments lacking the ability to bind Fcγ receptors are unable to mediate inflammation and can even inhibit IC_{nuc}-induced inflammation. Thus, antibodies, fragments or derivatives specific to an Ag_{nuc} and unable to bind to Fcγ receptors can be used to reduce inflammation associated with IC_{nuc}.

By blocking the activity of specific ICs of a given pathology, it addresses the causes of the development of the inflammatory response. In this respect, it differs from most current treatments that aim to address symptomatological consequences, most often by blocking inflammatory pathways, cytokines, or receptors involved in inflammation (Dinarello, 2010). This upstream operation could make the treatment more effective than other drugs on the market or under development.

In addition, the invention focuses on ICs generated during a pathology and is therefore only specific to this pathology. By blocking only the activity of the pathology-specific ICs, it allows other ICs to interact with FcRs and thus preserves the other physiological regulation processes of immune mechanisms.

In a first aspect, the present invention therefore concerns an antibody specific for a protein antigen capable of binding to nucleic acids, or an antigen-binding fragment or derivative of such an antibody, for its use as a medicament, characterized in that the antibody, fragment or derivative has a reduced FcγRIIA receptor binding capacity and/or an increased FcγRIIB receptor binding capacity.

The invention further concerns an antibody specific for a protein antigen capable of binding to nucleic acids, or an antigen-binding fragment or derivative thereof, for use in the treatment or prevention of inflammation, characterized in that the antibody, fragment or derivative has a reduced FcγRIIA receptor binding capacity and/or an increased FcγRIIB receptor binding capacity.

### DESCRIPTION OF THE FIGURES

FIG. 1A: Determination of B epitopes recognized by aTat12S and aTat7S Ab. The peptide Tat1-37 was adsorbed onto microtiter plates and the binding of both aTat12S and aTat7S Ab to Tat1-37-b was assessed by enzyme immunoassay.

FIG. 1B: Determination of B epitopes recognized by aTat12S and aTat7S antibodies. The peptide Tat37-57-b was adsorbed onto microtiter plates and the binding of both aTat12S and aTat7S Ab to Tat37-57-b was assessed by enzyme immunoassay.

FIG. 2: Assessment of the ability of Tat/aTat12S and Tat/aTat7S ICs to induce the inflammatory response of human PBMCs. Human PBMCs were incubated in vitro with 100 nM of free Tat, 100 nM of free aTat12S, 100 nM of free aTat7S, Tat/aTat12S (100 nM each), and Tat/aTat7S (100 nM each), respectively. After 18 hours of incubation, the supernatants were collected and the presence of IL-6 was assessed by enzyme immunoassay.

FIG. 3: Assessment of the ability of Ncp/aNcp2 and Ncp/aNcp15 ICs to induce the inflammatory response of human PBMCs. Human PBMCs were incubated in vitro with 30 nM of free Ncp, 30 nM of free aNcp2, 30 nM of free aNcp15, Ncp/aNcp2 (30 nM each), and Ncp/aNcp15 (30 nM each), respectively. After 18 hours of incubation, the supernatants were collected and the presence of IL-6 was assessed by enzyme immunoassay.

FIG. 4: The Fab-aNcp15 and mutated hcaNcp15Fc possess the ability to bind the Ncp protein. The chimeric antibody hcaNcp15 was adsorbed onto microtiter plates. A fixed concentration of N-biotinylated protein (178 pM) was incubated in the wells of the plate in the presence or absence of hcaNcp15 or Fab-Ncp15 or mutated hcaNcp15Fc serial dilutions. After overnight incubation at 4° C., the plate was washed and streptavidin coupled to peroxidase was added. After 30 minutes, washings were carried out and a colorimetric substrate (ABTS) was added to measure the presence of biotinylated Ncp.

FIG. 5: The ability of Ncp/aNcp ICs to induce the inflammatory response of human PBMCs is impaired when the Ab included in the complex lacks the Fc region. Human PBMCs were incubated in vitro with Ncp (30 nM), Fab-aNcp15 (333 nM), Ncp/hcaNcp15 (30 nM for each protein) and Ncp/Fab-aNcp15 (30 nM for Ncp, 333 nM for Fab), respectively. After 18 hours of incubation, the supernatants were collected and the presence of IL-6 was assessed by enzyme immunoassay.

FIG. 6: The ability of Ncp/aNcp ICs to induce the inflammatory response of human PBMCs is impaired when the Ab included in the complex is mutated in its Fc region so that it is no longer able to bind Fcγ receptors. Human PBMCs were incubated in vitro with Ncp (30 nM), mutated hcaNcp15Fc (30 nM), Ncp/hcaNcp15 (30 nM for each protein) and Ncp/mutated hcaNcp15Fc (30 nM for each protein), respectively. After 18 hours of incubation, the supernatants were collected and the presence of IL-6 was assessed by enzyme immunoassay.

FIG. 7: The ability of an aNcp to induce the inflammatory response of human PBMCs is impaired by an Ab derivative lacking the Fc region. Human PBMCs were incubated in vitro with Ncp (30 nM), Ncp/hcaNcp15 (30 nM for each protein), Ncp/Fab-aNcp15 (30 nM for Ncp, 333 nM for Fab), Ncp/hcaNcp15/Fab-aNcp15 (30 nM for the first two proteins, 333 nM for Fab, respectively). After 18 hours of incubation, the supernatants were collected and the presence of IL-6 was assessed by enzyme immunoassay.

FIG. 8: The ability of an aNcp to induce the inflammatory response of human PBMCs in the presence of Ncp is impaired by an Ab derivative mutated in its ability to bind RFcγs. Human PBMCs were incubated in vitro with Ncp (10 nM), Ncp/hcaNcp15 (10 nM for Ncp, 10 nM for the Ab), Ncp/mutated hcaNcp15Fc (10 nM for Ncp, 30 nM for the Ab), Ncp/hcaNcp15/mutated hcaNcp15Fc (10 nM for Ncp, 10 nM for hcaNcp15, 30 nM for mutated hcaNcp15Fc), respectively). After 18 hours of incubation, the supernatants were collected and the presence of IL-6 was assessed by enzyme immunoassay.

### DETAILED DESCRIPTION OF THE INVENTION

The invention concerns an antibody specific for a protein antigen capable of binding to nucleic acids, or to an antigen-binding fragment or derivative of such an antibody, for use as a medicament, characterized in that the antibody, fragment or derivative has a reduced FcγRIIA receptor binding capacity and/or an increased FcγRIIB receptor binding capacity.

It further concerns an antibody specific for a protein antigen capable of binding to nucleic acids, or an antigen-binding fragment or derivative of such an antibody, for its use in the treatment or prevention of inflammation, characterized in that the antibody, fragment or derivative has a reduced FcγRIIA receptor binding capacity and/or an increased FcγRIIB receptor binding capacity.

It further concerns the use of an antibody specific for a protein antigen capable of binding to nucleic acids, or of an antigen-binding fragment or derivative of such an antibody, for the preparation of a medicament intended for the treatment or prevention of inflammation, characterized in that the antibody, fragment or derivative has a reduced FcγRIIA receptor binding capacity and/or an increased FcγRIIB receptor binding capacity.

It further concerns the use of an antibody specific for a protein antigen capable of binding to nucleic acids, or of an antigen-binding fragment or derivative of such an antibody, for the treatment or prevention of inflammation, characterized in that the antibody, fragment or derivative has a reduced FcγRIIA receptor binding capacity and/or an increased FcγRIIB receptor binding capacity.

It further concerns a pharmaceutical composition comprising an antibody specific for a protein antigen capable of binding to nucleic acids, or an antigen-binding fragment or derivative of such an antibody, for its use in the treatment or prevention of inflammation, characterized in that the antibody, fragment or derivative has a reduced FcγRIIA receptor binding capacity and/or an increased FcγRIIB receptor binding capacity. It further concerns a method of treating or preventing inflammation in a subject in need thereof, comprising administering an effective amount of an antibody specific for a protein antigen capable of binding to nucleic acids, or an antigen-binding fragment or derivative thereof, characterized in that the antibody, fragment or derivative has a reduced FcγRIIA-receptor binding capacity and/or an increased FcγRIIB-receptor binding capacity.

### Features of the Antibody, Fragment or Derivative

The present invention is based on the therapeutic use of an antibody specific for a protein antigen capable of binding to nucleic acids.

The expression ‘antibody’ or ‘Ab’ or ‘immunoglobulin’ is understood to mean a molecule comprising at least one domain for binding to a given antigen and a constant domain comprising an Fc fragment capable of binding to FcR receptors. In most mammals, such as humans and mice, an antibody is composed of 4 polypeptide chains: 2 heavy chains and 2 light chains linked together by a variable number of disulfide bridges providing flexibility to the molecule. Each light chain consists of a constant domain (CL) and a variable domain (VL); the heavy chains consist of a variable domain (VH) and 3 or 4 constant domains (CH1 to CH3 or CH1 to CH4) depending on the isotype of the antibody. In some rare mammals, like camelids such as camels and llamas, and in sharks, antibodies consist of only two heavy chains, each heavy chain comprising a variable domain (called VhH in camelids and V-NAR in sharks) and a constant region (Holliger & Hudson, 2005).

Variable domains are involved in antigen recognition, while constant domains are involved in the biological, pharmacokinetic and effector properties of the antibody.

The variable region differs from one antibody to another. Indeed, the genes coding for the heavy and light chains of the antibodies are generated by recombination of three and two distinct gene segments, respectively, called VH, DH and JH-CH for the heavy chain and VL and JL-CL for the light chain. The CH and CL segments do not participate in recombination and form the constant regions of the heavy and light chains, respectively. The recombinations of the VH-DH-JH and VL-JL segments form the variable regions of the heavy and light chains, respectively. The VH and VL regions each have 3 hyper-variable zones or complementarity determining regions (CDR), called CDR1, CDR2 and CDR3, the CDR3 region being the most variable, since it is located at the recombination zone. These three CDR regions, and particularly the CDR3 region, are found in the part of the antibody that will be in contact with the antigen and are therefore very important for the recognition of the antigen. Thus, the vast majority of antibodies conserving the three CDR regions of each of the heavy and light chains of an antibody retain the antigenic specificity of the original antibody. In a certain number of cases, an antibody that retains only one of the CDRs, especially CDR3, also retains the specificity of the original antibody. The CDR1, CDR2 and CDR3 regions are each preceded by the FR1, FR2 and FR3 regions, respectively, corresponding to the framework regions (FR) which vary the least from one VH or VL segment to another. The CDR3 region is also followed by an FR4 framework region.

An antibody, fragment or derivative which ‘binds’ to an antigen of interest is an antibody, a fragment or derivative which binds to the antigen with sufficient affinity that the antibody is useful as a diagnostic and/or therapeutic agent to target the antigen in circulating form or expressed by a cell or tissue, and does not significantly react with other antigens. The extent of the binding of the antibody, fragment or derivative to a ‘non-target’ antigen will be less than approximately 10% of the binding of the antibody to its target antigen, as determined by fluorescence activated cell sorting analysis (FACS) or radioimmunoprecipitation analysis (RIPA). With regard to the binding of an antibody, fragment or derivative to a target antigen, the expressions ‘specific binding’ or ‘specifically binds to’ or is ‘specific for’ a particular antigen or epitope of a particular antigen mean a binding that is measurably different from a non-specific interaction. Specific binding can be measured, for example, by measuring binding to the antigen relative to binding to a control antigen, which is usually an antigen of similar structure that has no binding activity. For example, specific binding can be determined by competing with a control antigen that is similar to the target, for example by measuring binding to the labelled target antigen in the presence or absence of excess unlabelled target antigen. In this case, binding is considered specific if binding of the labelled target is competitively inhibited by an excess of unlabelled target. It may especially be considered that there is ‘specific binding’ or that the antibody, fragment or derivative ‘specifically binds’ to or is ‘specific’ for a particular antigen or epitope on an antigen, for example, if the antibody, fragment or derivative has a KD for the target of at most about 10⁻⁶ M, in a variant at most about 10⁻⁷ M, in a variant at most about 10⁻⁸ M, in a variant at most about 10.9 M, in a variant at most about 10⁻¹⁰ M, in a variant at most about 10⁻¹¹ M, in a variant at most about 10⁻¹² M, or even less. In some embodiments, the term ‘specific binding’ refers to a binding in which the antibody, fragment or derivative binds to a particular antigen or epitope on an antigen without substantially binding to another antigen or epitope. In certain embodiments, an antibody, fragment or derivative that binds to an Ag_{nuc} has a dissociation constant (KD) of ≤1 μM, ≤100 nM, ≤10 nM, ≤1 nM, or ≤0.1 nM.

The term ‘KD’ used here means a dissociation constant of a specific antibody-antigen interaction and is used as an indicator for measuring the affinity of an antibody for an antigen. A lower KD means a higher affinity of an antibody for an antigen.

Unlike variable domains whose sequence varies greatly from one antibody to another, constant domains are characterized by a very close amino acid sequence from one antibody to another, characteristic of the species and isotype, possibly with some somatic mutations. The Fc fragment is naturally composed of the constant region of the heavy chain excluding the CH1 domain, i.e. the lower hinge region and the CH2 and CH3 or CH2 to CH4 constant domains (depending on the isotype). In human IgG1, the complete Fc fragment consists of the C-terminal portion of the heavy chain from the cysteine residue at position 226 (C226), the numbering of the amino acid residues in the Fc fragment throughout the present description being that of the EU index described in the publications Edelman et al. —1969 and Kabat et al.- 1991 (Edelman et al., 1969; Kabat E. A., Wu T. T., Perry H. M. Foeller C., 1991). The corresponding Fc fragments of other types of immunoglobulins can be easily identified by the person skilled in the art by sequence alignments.

The Fc fragment of IgG is glycosylated at the CH2 domain with the presence, on each of the 2 heavy chains, of an N-glycan linked to the asparagine residue at position 297 (ASN 297).

The following binding domains, located in Fc, are important for the biological properties of the antibody:
FcRn receptor binding domain, involved in the pharmacokinetic properties (half-life in vivo) of the antibody:

Various data suggest that some residues located at the interface of the CH2 and CH3 domains are involved in binding to the FcRn receptor.
complement protein binding domain C1q, involved in the ‘complement-dependent cytotoxicity’ (CDC) response: located in the CH2 domain;
FcR receptor binding domain, involved in phagocytosis or antibody-dependent cellular cytotoxicity (ADCC) responses: located in the CH2 domain.

The antibodies can be of several isotypes, depending on the nature of their constant region: the constant regions γ, α, μ, ε and δ correspond, respectively, to immunoglobulins IgG, IgA, IgM, IgE and IgD. Advantageously, the monoclonal antibody present in a composition used as a medicament in the context of the invention is of the IgG isotype.

In the context of the invention, the antibody may be monoclonal or polyclonal.

In an advantageous embodiment, the antibody is monoclonal. ‘Monoclonal antibody’ or ‘monoclonal antibody composition’ is understood to mean a composition comprising antibody molecules having an identical and unique antigenic specificity. The antibody molecules present in the composition are likely to vary in their post-translational modifications, and especially in their glycosylation structures or isoelectric point, but have all been encoded by the same heavy and light chain sequences and therefore have the same protein sequence before any post-translational modification. Some differences in protein sequences, linked to post-translational modifications (such as, for example, cleavage of the C-terminal lysine of the heavy chain, deamidation of asparagine residues and/or isomerization of aspartate residues), may nevertheless exist between the different antibody molecules present in the composition.

In another embodiment, the antibody is polyclonal. The term ‘polyclonal antibody’ is understood to mean a mixture of antibodies recognizing different epitopes on a given antigen. It may be a polyclonal antibody purified from the serum of a subject immunized with the antigen of interest (this will then be referred to as ‘natural polyclonal’, whether the immunization is natural or human-induced) or a mixture of at least 2 (for example 2, 3, 4 or 5) monoclonal antibodies recognizing different epitopes on a given antigen (this will then be referred to as ‘synthetic polyclonal’). In the context of the invention, a natural or synthetic polyclonal antibody may be used, with preference for a synthetic polyclonal antibody.

For use in humans, the antibody, functional fragment or derivative thereof according to the invention is advantageously a chimeric or humanized antibody, in particular a chimeric antibody whose constant region of the heavy and light chains is of human origin.

The term ‘chimeric’ antibody is intended to designate an antibody which contains a natural variable region (light chain and heavy chain) derived from an antibody of a given species in association with the light chain and heavy chain constant regions of an antibody of a species heterologous to said given species. Advantageously, if the monoclonal antibody composition for its use as a medicament according to the invention comprises a chimeric monoclonal antibody, the latter comprises human constant regions. Starting from a non-human antibody, a chimeric antibody can be prepared using genetic recombination techniques well known to the person skilled in the art. For example, the chimeric antibody may be produced by cloning for the heavy chain and the light chain a recombinant DNA comprising a promoter and a sequence coding for the variable region of the non-human antibody, and a sequence coding for the constant region of a human antibody. For the methods for preparing chimeric antibodies, reference may be made, for example, to the documents (Verhoeyen et al., 1988; Verhoeyen & Riechmann, 1988).

The term “humanized” antibody is intended to designate an antibody which contains CDR regions derived from an antibody of non-human origin, the other parts of the antibody molecule being derived from one (or more) human antibodies. In addition, some of the residues of the skeleton segments (called FR) can be modified to retain the binding affinity (Jones et al., 1986; Riechmann et al., 1988; Verhoeyen et al., 1988). The humanized antibodies according to the invention can be prepared by techniques known to the person skilled in the art such as CDR grafting, resurfacing, SuperHumanization, human string content, FR libraries, guided selection, FR shuffling and humaneering technologies, as summarized in the review by Almagro et al. (Almagro & Fransson, 2008).

The terms ‘whole antibody’, ‘intact antibody’ or ‘full-length antibody’ are used interchangeably to designate an antibody in its substantially intact form, as opposed to an antibody fragment. Specifically, ‘whole antibodies’ as used herein include those with heavy and light chains comprising an Fc domain. The constant domains may be native sequence constant domains (for example, human native sequence constant domains) or amino acid sequence variants thereof.

The term ‘antigen-binding fragment of an antibody’ is understood to mean an antibody fragment which retains the antigen binding domain and therefore has the same antigenic specificity as the original antibody. Non-limiting examples of fragments include F(ab′)2, Fab, Fab′, ScFv, FV, VhH, and V-NAR fragments. The structures of these fragments and the methods for obtaining them are known to the person skilled in the art (Holliger & Hudson, 2005).

The term ‘antigen-binding derivative of an antibody’ is understood to mean a complex comprising several fragments of an antibody arranged in a non-natural form. Non-limiting examples of fragments include derivatives of ScFv, Bis-scFv, scFv-Fc, Fab₂, Fab₃, minibodies, diabodies, triabodies and tetrabodies. The structures of these derivatives and the methods for obtaining them are also known to the person skilled in the art (Holliger & Hudson, 2005).

In the presence of their antigen, antibodies form immune complexes (ICs), i.e., antigen-antibody aggregates comprising several antibodies bound to the surface of one or more antigen molecules.

The antibody, fragment or derivative used in the invention has a reduced FcγRIIA receptor binding capacity and/or an increased FcγRIIB receptor binding capacity.

Antibodies, especially IgGs, are bifunctional molecules. They interact with the different receptors of the Fcγ domain (FcγRs) via their Fc domain and bind antigens via their two Fab domains. The ICs formed allow better management of antigens by immune cells expressing FcγRs on their surface. The FcγRs are classified into two functional groups, activator FcγRs (FcγRI, FcγRIIA, FcγRIIC, FcγRIIIA) and inhibitor FcγRs (FcγRIIB). Among the activator FcγRs, FcγRI is a high affinity receptor capable of binding antibodies in monomeric form, while FcγRIIA, FcγRIIC, and FcγRIIIA are low affinity receptors that bind mainly ICs. The inhibitory FcγRIIB receptor is also a low affinity receptor, binding mainly ICs.

An Antibody, Fragment, or Derivative with Reduced FcγRIIA Receptor Binding Capacity

In an advantageous embodiment, an antibody, fragment or derivative having a reduced FcγRIIA receptor binding capacity is used.

The antibody, fragment or derivative may also have a reduced capacity for binding to the FcγRIIIA receptor. It may also have a reduced capacity for binding to all FcγRs.

‘Reduced binding capacity’ to one or more FcγRs is understood to mean that the binding of the antibody, fragment or derivative to FcγR is weaker than that of an antibody with the same variable regions, but whose constant regions are natural (native antibody). Advantageously, the FcγR-binding capacity is lower by a factor of at least 2, at least 5, at least 10, or sometimes even at least 25, at least 50, at least 75, or even at least 100, than that of the native antibody.

In this case, a fragment or derivative without Fc domain can advantageously be used. Indeed, the absence of Fc domain prevents any binding to FcγRs, and thus especially to FcγRIIA, and to FcγRIIIA.

Fragments without Fc domain especially include F(ab′)2, Fab, Fab′, Fv, VhH, and V-NAR fragments.

Derivatives without Fc domain especially include ScFv, Bis-scFv, Fab₂, Fab₃, diabody, triabody and tetrabody derivatives.

Thus, in a preferred embodiment, a fragment or derivative without Fc domain, advantageously selected from F(ab′)2, F(ab′), Fab, Fab′, Fv, VhH, V-NAR, ScFv, Bis-scFv, Fab₂, Fab₃, diabodies, triabodies and tetrabodies is used.

Moreover, as previously indicated, the CH2 domain of IgG isotype antibodies is known to comprise the FcγRs-binding domain. Therefore, it is also possible to use a fragment or derivative that has part of the Fc domain but does not include the CH2 domain, such as minibodies).

To reduce the FcγRIIA binding capacity, it is also possible to use a whole antibody of the IgG4 isotype, this isotype binding FcγRIIA very weakly. It is also possible to use a whole antibody of IgG2-IgG4 cross isotype comprising the CH1 domain and the hinge region of IgG2 isotype and the CH2 and CH3 domains of IgG4 isotype (Rother et al., 2007).

To reduce the binding capacity to FcγRIIA and possibly also to FcγRIIIA (or even all FcγRs), it is also possible to use a whole antibody of the IgG isotype or a derivative thereof with an Fc domain (such as scFv-Fc), whose Fcγ domain has been modified to reduce or abolish its binding to the receptors of interest (FcγRIIA alone, FcγRIIA and FcγRIIIA, or all FcγRs).

A first Fc domain modification strategy to reduce or abolish binding to the FcγRs of interest consists of inserting one or more mutations in the Fc domain. Indeed, many mutations or combinations of mutations of the Fcγ domain are known to reduce or abolish binding to some or all FcγRs.

Among the mutations and combinations of mutations of particular interest, mention may be made (Chenoweth et al., 2020; Vafa et al., 2014; Wang et al., 2018):
for IgG1: point mutations E233P, L234A, L234F, L235A, L235E, L236E, D265A, P331S, or any combination of these mutations, especially combinations L234A/L235A and L234A/L235E/P331S, which reduce or eliminate FcγR binding.

The mutations E233P, F234V, L235A and D265A eliminate the effector functions. The mutations and combinations of mutations L235E and L234A/L235A reduce effector functions.
for IgG2: point mutations V234A, G237A, P238S, H268A, H268Q, V309L, A330S, P331S, or any combination thereof, especially combinations H268Q/V309L/A330S/P331S and V234A/G237A/P238S/H268A/V309L/A330S/P331S, which reduce FcγR binding;
for IgG4: point mutations F234A, L235A, and L235E or combinations F234A/L235A, H268Q/V309L/A330S/P331S (weak interaction with FcγRI, elimination of interaction with FcγRIIA FcγRIIIA), S228P/L234A/L235A (very weak interaction with FcγRI, FcγRIIA and FcγRIIIA) which reduce FcγR binding.

In a preferred embodiment, the antibody used in the invention is of the IgG1 isotype and its Fc domain comprises a combination of the 4 mutations E233P, F234V, L235A, and D265A.

A second Fc domain modification strategy for reducing or abolishing FcγR binding consists of altering Fc domain glycosylation according to embodiments known to the person skilled in the art (Strohl & Strohl, 2012). This alteration can be achieved using cells with variable post-translational capacities, by the removal of the glycosylation site, or by enzymatic deglycosylation of the antibody.

The glycosylation site of the Fc domain can especially be deleted by replacing the asparagine residue to which N-glycans are bound with another amino acid (for example, for an IgG1, by an N297A, N297Q or N297G mutation (elimination of interaction with FcγRIIA and FcγRIIIA, reduced interaction with FcγRI); see (Wang et al., 2018).

To obtain a non-glycosylated antibody, it is also possible to produce the antibody in a host without an N-glycosylation system, such as, for example, bacteria, which are naturally lacking an N-glycosylation system, or any other host that normally has an N-glycosylation system but has been modified to no longer N-glycosylate proteins, such as yeasts, plants, insect cells or mammalian cells.

To obtain a non-glycosylated antibody, it is also possible to deglycosylate the antibody a) enzymatically, for example by using PNGase F (peptide-N4-(acetyl-B-glucosaminyl)-asparagine amidase, EC 3.5.1.52), endoglycosidase such as endo-alpha-N-acetyl-galactosaminidase, endoglycosidase F1, endoglycosidase F2, endoglycosidase F3, or endoglycosidase H or (b) chemically.

The modification strategies 1 and 2 presented above can potentially be combined (mutation(s) in Fc reducing binding to the FcγRs of interest and absence of glycosylation). In return, when glycosylation is not suppressed, producing the whole antibody or derivative with an Fc domain should be avoided under conditions resulting in glycosylation known to increase the binding capacity to the FcγRs of interest (producing an antibody or derivative whose Fc domain is glycosylated with a low fucosylation will especially be avoided, this greatly increasing the binding to FcγRIIIA).

An Antibody, Fragment, or Derivative with Increased FcγRIIB Receptor Binding Capacity

In another advantageous embodiment, an antibody, fragment or derivative having an increased FcγRIIB receptor binding capacity is used.

‘Increased FcγRIIB receptor binding capacity’ is understood to mean that the binding of the antibody, fragment or derivative to the FcγRIIB receptor is stronger than that of an antibody with the same variable regions, but whose constant regions are natural (native antibody); advantageously, the FcγRIIB-binding capacity is greater by a factor of at least 2, at least 5, at least 10, or sometimes even at least 25, at least 50, at least 75, or even at least 100, than that of the native antibody.

In this case, a whole antibody of the IgG isotype which possesses one or more mutations in the Fc domain significantly increasing its binding to the inhibitory FcγRIIB receptor will advantageously be used.

Appropriate modifications of the Fc domain to increase receptor FcγRIIB-binding capacity especially include combinations of S267E/L328F (or “SELF”, but maintain a high affinity for FcγRIIA-R), N325S/L328F (concomitant reduction of interaction with FcγRIIIA) and P238D/E233D/G237D/H268D/P271G/A3301 (or “V12”, no modification of interaction with FcγRIIa-R131) (Chenoweth et al., 2020; Mimoto et al., 2013; Wang et al., 2018).

### Antigen Recognized by the Antibody, Fragment or Derivative

The antibody, fragment or derivative used in the invention specifically recognizes a protein antigen capable of binding to nucleic acids (Ag_{nuc}).

Preferably, the protein antigen specifically recognized by the antibody, fragment or derivative used in the invention has one of the following features:
a) it is capable of binding to nucleic acids alone, that is to say without being complexed to another molecule (other protein especially);
b) it is capable of binding to nucleic acids without specificity for a given nucleic sequence;
c) It is capable of binding to nucleic acids more efficiently at pH 7.0 to 7.5 than at acidic pH (below 7.0);
d) it comprises one or more positively-charged accessible region(s).

In the case of globular proteins, Ag_{nuc} preferably comprises one or more positively charged surface regions; in the case of naturally unfolded proteins/peptides, the Ag_{nuc} preferably comprises one or more region(s) in the primary sequence containing positively charged amino acids;
e) it is also capable of binding to membrane heparan sulfate proteoglycans, preferably alone, without being complexed to another molecule (other protein especially); and
f) any combination of features (a) to (e).

In particular, the protein antigen specifically recognized by the antibody, fragment or derivative used in the invention may have one of the following combinations of features:
i. the combination of features a) and d);
ii. the combination of features a) and e);
iii. the combination of features b) and d);
iv. the combination of features b) and e);
v. the combination of features d) and e);
vi. the combination of features a), b) and d);
vii. the combination of features a), d) and e);
viii. the combination of features b), d) and e);
ix. the combination of features a), b), d) and e);
x. the combination of features b) to e); or
xi. the combination of features a) to e)

Feature e) (ability to bind to membrane heparan sulfate proteoglycans, preferably alone) is of particular interest insofar as one hypothesis concerning the mechanism by which immune complexes (ICs) between viral capsid proteins or ribo- or deoxyribonucleoproteins and nucleic acids cause hyperinflammation in vivo is that these ICs excessively activate effector cells by double binding:
binding the Fc domains of IC antibodies to Fcγ receptors, and
binding the antigen recognized by the antibodies (viral capsid proteins or ribo- or deoxyribonucleoproteins) to membrane heparan sulfate proteoglycans also present on the surface of effector cells.

This second binding would enhance the activator effect of binding between the Fc domains of antibodies and Fcγ receptors.

This ability to also bind membrane heparan sulfate proteoglycans, preferably alone, is present in many proteins having characteristic d), the membrane heparan sulfate proteoglycans being negatively charged.

When the Ag_{nuc} comprises feature d), the recognition of nucleic acids is also generally obtained without specificity for a given nucleic sequence (feature b), and a combination of features b) and d) is therefore also of particular interest.

When the Ag_{nuc} comprises characteristic d), the recognition of nucleic acids is also generally obtained without it being complexed to another molecule (feature a), and a combination of features a) and d) is therefore also of particular interest.

Three or four of the features a), b), d) and e) will also be present in many Ag_{nuc}, and a combination of the features a), b), and d), a combination of the features a), d) and e), a combination of the features b), d) and e), or a combination of a), b), d) and e) are also combinations of interest.

Since the binding between the antibody and its Ag_{nuc} must take place in vivo, it is preferably more effective at a pH of between 7.0 and 7.5 (feature c). Each of the above combinations can therefore also be combined with characteristic c).

Many protein antigens are capable of binding to nucleic acids. Such Ag_{nuc} are especially described in various publicly accessible databases, especially those described in Table 1 below:

TABLE 1
Databases describing Ag_{nuc}.

| Database | Description | Site |
|---|---|---|
| ENPD | Eukaryotic Nucleic acid | http://qinlab.sls.cuhk.edu.hk/ |
| | binding Protein Database | ENPD/ |
| RBPDB | RNA-binding proteins | http://rbpdb.ccbr.utoronto.ca/ |
| | | index.php |
| RBPbase | Eukaryotic RNA-binding | https://rbpbase.shiny.embl.de/ |
| | proteins | |
| hRBPome | Human RNA-binding proteins | http://caps.ncbs.res.in/ |
| | | hrbpome/ |
| CollecTF | Transcription factor binding | http://collectf.umbc.edu/ |
| | sites (TFBS) in the Bacteria | browse/home/ |

### Infectious Microorganism Antigens:

In an advantageous embodiment, the Ag_{nuc} that the antibody, fragment or derivative used in the invention specifically recognizes is selected from the proteins of infectious microorganisms capable of binding to nucleic acids. This type of antibody is used when the inflammation is due to infection by a microorganism.

The infectious microorganism protein capable of binding to nucleic acids is preferably internal to the infectious microorganism, in other words it is not on the surface of the infectious microorganism. It is, in addition or alternatively, preferably capable of binding to nucleic acids in native form, before any cleavage inducing a change in conformation.

In particular, the Ag_{nuc} that the antibody, fragment or derivative used in the invention specifically recognizes is advantageously selected from viral proteins capable of binding to the viral genome. Preferably, the Ag_{nuc} that the antibody, fragment or derivative used in the invention specifically recognizes is selected from the internal proteins of the virus, with the exception of the viral proteins located on the surface of the virus. Preferred viral Ag_{nuc} generally include viral capsid proteins, but also other viral proteins such as the transcriptional transactivator (Tat) and reverse transcriptase (RT, p66/p51) of human immunodeficiency virus 1 (HIV-1).

The term ‘capsid’ is understood to mean the shell which surrounds the viral genome. The protein capable of packaging the viral genome to form the capsid is called ‘capsid protein’ or ‘nucleocapsid protein’. Indeed, the term ‘nucleocapsid’ designates, in principle, the viral genome surrounded by the capsid, but is sometimes also used to designate the capsid. Other proteins may associate with the capsid, such as matrix proteins, or the envelope around the capsid of enveloped viruses, but are not considered capsid proteins.

Among the viral capsid proteins more particularly of interest, mention may be made of:
HIV-1 capsid protein, also called p24 (amino acid sequence corresponding to GenBank accession no. NP_057850.1 (residues 133-363), GenBank version 241 of 15 Dec. 2020);
the capsid protein of the severe acute respiratory syndrome virus 2 (‘SARS-COV-2’) coronavirus, responsible for COVID-19, amino acid sequence corresponding to GenBank accession no. YP_009724397.2, GenBank version 241 of 15 Dec. 2020),
influenza virus capsid protein (amino acid sequence corresponding to GenBank accession no. AAA43798.1, GenBank version 241 of 15 Dec. 2020);
hepatitis B virus capsid protein (HBV, amino acid sequence corresponding to GenBank accession no. AXM44956.1, GenBank version 241 of 15 Dec. 2020);
hepatitis E virus capsid protein (HEV, amino acid sequence corresponding to GenBank accession no. P03314.1 (residues 1 to 101), GenBank version 241 of 15 Dec. 2020);
human papillomavirus capsid protein (HPV, amino acid sequence corresponds to GenBank accession no. AQZ41126.1, GenBank version 241 of 15 Dec. 2020);
dengue virus capsid protein (amino acid sequence corresponding to GenBank accession no. AAW51422.1 (residues 5 to 114), GenBank version 241 of 15 Dec. 2020.

Indeed, these proteins (capsid proteins above and Tat or RT of HIV-1) are known to generate an antibody response in patients infected with these viruses and, in some cases, the presence of immune complexes (ICs) formed by these antigens and antibodies specifically recognizing them is also known to be associated with pathogenesis (Cafaro et al., 2019; Carter et al., 2000; Fenouillet et al., 1993; Hashida et al., 1997; Hu, 2002; Kamar et al., 2017; Ni et al., 2020; Rumbaugh et al., 2013).

The ICs of the intracellular protein of Yersinia enterocolitica 0.3 (IcP-Ye) are also associated with certain types of chronic arthritis, and antibodies, fragments or derivatives specific to this protein can therefore also be used.

Monoclonal antibodies specifically recognizing the transcriptional transactivator (Tat) of HIV-1 have been described in vaccine strategies (Cafaro et al., 2019) and are available on the market, such as, for example:
‘Purified anti-HIV TAT’, mouse monoclonal antibody, Ref.: 919001, sold by BioLegend.
‘Recombinant Human anti-HIV-1 Tat Antibody (B1E3)’, recombinant human antibody, Ref.: MRO-4310CQ, sold by Creative Biolabs.

Monoclonal antibodies specifically recognizing HIV-1 reverse transcriptase (RT, p66/p51) are commercially available, such as, for example: ‘Anti-HIV1-RT antibody’, mouse monoclonal antibody, Ref.: ABIN933463, sold by Antibodies-online.

Monoclonal antibodies specifically recognizing HIV-1 nucleocapsid (p24) are commercially available, such as, for example:
‘Anti-HIV1 p24 antibody [39/5.4A]’, mouse monoclonal antibody, Ref: ab9071, sold by Abcam.
‘Anti-HIV-1 p24 Therapeutic Antibody (19H2)’, recombinant human antibody (IgG), Ref: MRO-082LC, sold by Creative Biolabs.

Monoclonal antibodies specifically recognizing the nucleocapsid of SARS-COV-2 virus are available on the market such as, for example:
‘Nucleocapsid Antibody’, mouse monoclonal antibody, Ref.: MAB104741, sold by R&D SYSTEMS.
‘SARS-COV-2 Nucleocapsid Antibody (bcn11)’, humanized antibody, Ref: MA5-35953, sold by Thermo Fisher Scientific.

Monoclonal antibodies specifically recognizing the nucleocapsid of influenza virus are available on the market such as, for example: ‘Recombinant Mouse Anti-Influenza A virus Nucleocapsid protein Antibody (CBI49YJ)’, mouse monoclonal antibody, Ref.: HPAB-0295-YJ sold by Creative Biolabs.

Monoclonal antibodies specifically recognizing the HBV nucleocapsid are commercially available, such as, for example: ‘Recombinant Hepatitis B Core Antigen (rHBcAg)’, mouse monoclonal antibody, Ref.: bsm-2000M, sold by BiossAntibodies.

Monoclonal antibodies specifically recognizing the HEV nucleocapsid are commercially available, such as, for example: ‘HEV/Hepatitis E Virus Monoclonal Antibody’, mouse monoclonal antibody, Ref.: LS-C67675, sold by LifeSpan Biosciences.

Monoclonal antibodies specifically recognizing the HPV nucleocapsid are commercially available, such as, for example: ‘Human Papilloma Virus L1 Antibody’, mouse monoclonal antibody, Ref.: MBS320499, sold by MyBioSource.com (specific for the major structural protein of the L1 capsid).

Monoclonal antibodies specifically recognizing the intracellular protein of Yersinia enterocolitica 0.3 (ICP-Ye) such as, for example mouse monoclonal Ab, Ref.: 2D8-P, sold by PROGEN.

Other monoclonal antibodies that recognise Ag_{nuc} of infectious microorganisms (especially viruses, such as HIV-1 transcriptional transactivator (Tat) or reverse transcriptase (RT) or viral nucleocapsid proteins, in particular SARS-COV-2, influenza, HBV, HEV, and HPV) can be generated by the person skilled in the art on the basis of the sequences of these proteins, using routine methods, for example the one described in the examples.

On the basis of such monoclonal antibodies, the person skilled in the art will be able to generate fragments or derivatives without Fc domain, or with an Fc domain modified so as not to bind to FcγRIIA and FcγRIIIA (activating receptors), so as not to bind to any FcγR, or even to bind preferentially to FcγRIIb (inhibitor receptor).

### Self Antigens:

In another advantageous embodiment, the Ag_{nuc} that the antibody, fragment or derivative used in the invention specifically recognizes is selected from the self proteins of the subject to be treated. This type of antibody is used when the inflammation is due to an autoimmune disease.

The self protein capable of binding to nucleic acids is preferably an intracellular protein. It is, in addition or alternatively, preferably capable of binding to nucleic acids in native form, before any cleavage inducing a change in conformation.

The Ag_{nuc} may especially be selected from ribonucleoproteins and deoxyribonucleoproteins of the subject to be treated.

Among ribonucleoproteins, mention may be made more particularly of the following proteins:
the RibP protein (amino acid sequence corresponding to GenBank accession No. NP_000993.1 (P0 subunit), NP_000994.1 (P1 subunit) and NP_000995.1 (P2 subunit), GenBank version 241 of 15 Dec. 2020; RibP ICs are associated with the pathogenesis of systemic lupus erythematosus, see (Choi et al., 2020)
SnRNPs, comprising protein A, protein C and the protein at 70K (amino acid sequences corresponding to GenBank accession nos. NP_004587.1, NP_003084.1 and NP_003080.2, GenBank version 241 of 15 Dec. 2020). ICs of snRNPs A, C and 70K are associated with the pathogenesis of systemic lupus erythematosus, rheumatoid arthritis, and systemic sclerosis, see (Pisetsky & Lipsky, 2020) and proteins B, B′ and D (amino acid sequences corresponding to GenBank accession nos. NP_003082.1 and NP_008869.1, version 241 of GenBank of 15 Dec. 2020; ICs of snRNPs B, B′ and D are associated with the pathogenesis of systemic lupus erythematosus, see (Pisetsky & Lipsky, 2020),
the Ro60 protein (amino acid sequence corresponding to GenBank accession no. NP_001035828.1, GenBank version 241 of 15 Dec. 2020); Ro60 ICs are associated with the pathogenesis of systemic lupus erythematosus, primary Sjögren syndrome, and systemic sclerosis, see (Schulte-Pelkum et al., 2009),
the Ro52 protein (amino acid sequence corresponding to GenBank accession no. NP_003132.2, GenBank version 241 of 15 Dec. 2020); the ICs of Ro52 are associated with the pathogenesis of systemic lupus erythematosus, see (Schulte-Pelkum et al., 2009), and
the La protein (lupus antigen, amino acid sequence corresponding to GenBank accession no. NP_001281074.1, GenBank version 241 of 15 Dec. 2020); La ICs are associated with the pathogenesis of systemic lupus erythematosus, see (To & Petri, 2005).

Among the deoxyribonucleoproteins, mention may be made more particularly of histones (for example, the amino acid sequence corresponding to GenBank accession no. NP_001035807.1 (histone 2A), CAA41051.1 (histone 2-B), AAN39284.1 (histone 3), NP_003486.1 (histone 4), GenBank version 241 of 15 Dec. 2020); histone ICs are associated with the pathogenesis of systemic lupus erythematosus, see (Ghiggeri et al., 2019).

Polyclonal antibodies specifically recognizing the RibP protein are available on the market such as, for example: ‘Ribosomal P Antigen antibody’, rabbit polyclonal antibody, Ref.: GTX39242, sold by GeneTex.

Monoclonal antibodies specifically recognizing snRNPs are available on the market such as, for example: ‘Anti-SM BB’ Proteins (Human autoantigens) Monoclonal Antibody’, mouse monoclonal antibody, Ref.: 03-57029 sold by American Research Products, Inc.™.

Monoclonal antibodies specifically recognizing the Ro60 protein are available on the market such as, for example: ‘Anti-Ro60 (SS-A) Protein Monoclonal Antibody’, clone 1D8, mouse monoclonal antibody, Ref.: 03-57039 sold by American Research Products, Inc.™.

Monoclonal antibodies specifically recognizing the Ro52 protein are available on the market such as, for example: ‘Anti-TRIM21 Mouse mAb’, mouse monoclonal antibody, Ref.: MBS475588, sold by MyBioSource.com.

Polyclonal antibodies specifically recognizing the Lupus antigen (La) are available on the market such as, for example: ‘Anti-Lupus La protein SSB Antibody’, rabbit polyclonal antibody, Ref.: A00705, marketed by BosterBio.

Monoclonal antibodies specifically recognizing histones are available on the market such as, for example: ‘Histone Monoclonal Antibody’, mouse monoclonal antibody, Ref.: LS-C68011, sold by LifeSpan Biosciences.

Other monoclonal antibodies recognizing the Ag_{nuc} of self proteins (especially those described above) can be generated by the person skilled in the art on the basis of the sequences of these proteins using routine methods, e.g., the one described in the examples.

On the basis of such monoclonal antibodies, the person skilled in the art will be able to generate fragments or derivatives without Fc domain, or with an Fc domain modified so as not to bind to FcγRIIA and FcγRIIIA (activating receptors), so as not to bind to any FcγR, or even to bind preferentially to FcγRIIb (inhibitor receptor).

### Inflammation

The antibodies, fragments and derivatives described above are useful in the treatment of inflammation, and in particular in the treatment of inflammation due to the presence of immune complexes (ICs) formed of an Ag_{nuc} and antibodies specifically recognizing this Ag_{nuc}.

The diseases in which these ICs are most commonly observed and have been associated with disease pathogenesis are mainly:
infections by microorganisms (especially viral infections), in which ICs formed between an Ag_{nuc} of the infectious microorganism (especially viral capsid proteins, which generate strong antibody responses) and antibodies that recognize this Ag_{nuc} can induce acute inflammation that can lead to ‘cytokine storms’ (high secretion of inflammatory cytokines with deleterious effects on tissues) described in severe forms of COVID-19 and influenza, or chronic inflammation, and
autoimmune diseases, where the presence of ICs formed between a self Ag_{nuc} and antibodies recognizing this Ag_{nuc} can also induce acute or chronic inflammation.

Thus, the antibodies, fragments and derivatives described above are particularly useful in the treatment of inflammation due to:
a) infection by a microorganism; or
b) an autoimmune disease.

### Inflammation Due to Infection by a Microorganism

In the case of inflammation due to infection by a microorganism, an antibody, fragment or derivative as described herein that binds specifically to an Ag_{nuc} of the microorganism responsible for the infection will be used.

Advantageously, the infection is a viral infection, since it is in this type of infection that the greatest number of ICs associated with the pathogenesis of the infection have been observed, and then an antibody, a fragment or derivative as described herein is used that specifically binds to an Ag_{nuc} (in particular, the capsid protein) of the virus responsible for the infection.

The viral infection is advantageously selected from COVID-19, influenza, AIDS, hepatitis B, hepatitis E, human papillomavirus infections and dengue.

Thus, in the case of inflammation during COVID-19 due to the SARS-COV-2 virus (especially in the case of the severe form of COVID-19), it is possible to use an antibody, a fragment or a derivative as described here which specifically binds to the capsid protein of the SARS-COV-2 virus.

In the case of inflammation during influenza due to the influenza virus, an antibody, fragment or derivative as described here which specifically binds to the capsid protein of the influenza virus may be used.

In the case of inflammation during AIDS due to the HIV-1 virus, it is possible to use an antibody, a fragment or a derivative as described here which binds specifically to the capsid protein (p24), the transcriptional transactivator (Tat) or the reverse transcriptase (RT) of HIV-1.

In the case of inflammation during hepatitis B due to the HBV virus, an antibody, fragment or derivative as described herein which binds specifically to the capsid protein of the HBV virus may be used.

In the case of inflammation during hepatitis E due to the HBV virus, an antibody, fragment or derivative as described herein which binds specifically to the capsid protein of the HEV virus may be used.

In the case of inflammation during infections by the HPV virus, an antibody, fragment or derivative as described herein which binds specifically to the capsid protein of the HPV virus may be used.

In the case of inflammation during dengue due to the dengue virus, an antibody, fragment or derivative as described herein which binds specifically to the capsid protein of the dengue virus may be used.

However, inflammation can also be due to bacterial or parasitic infection. In this case, an antibody, fragment or derivative as described herein which binds specifically to an Ag_{nuc} of the bacterium or parasite responsible for the infection will be used. For example, in the case of chronic arthritis due to an infection by Yersinia enterocolitica 0.3, it is possible to use an antibody, a fragment or a derivative as described here which binds specifically to the intracellular protein of Yersinia enterocolitica 0.3 (ICP-Ye).

### Inflammation Due to Autoimmune Disease

In the case of inflammation due to infection due to autoimmune disease, an antibody, fragment or derivative as described herein which binds specifically to a self Ag_{nuc} of the patient to be treated will be used.

The autoimmune disease is advantageously selected from rheumatoid arthritis, Kawasaki disease, systemic lupus erythematosus, systemic sclerosis and primary Sjögren syndrome because these are the autoimmune diseases in which the greatest number of ICs associated with the pathogenesis of the disease have been observed.

For example, in the case of rheumatoid arthritis, it is especially possible to use an antibody, a fragment or a derivative as described here which binds specifically to a protein selected from the snRNPs A, C or 70K.

In the case of systemic lupus erythematosus, it is especially possible to use an antibody, a fragment or a derivative as described here which binds specifically to a protein selected from the RibP protein, histones, snRNPs A, B, B′, C, D and 70K, and Ro60 protein, and the Ro52 protein.

In the case of systemic sclerosis, it will be possible in particular to use an antibody, a fragment or a derivative as described here which binds specifically to a protein selected from the Ro60 protein and the snRNPs A, C or 70K.

In the case of primary Sjögren syndrome, an antibody, fragment or derivative as described herein which binds specifically to the Ro60 protein will especially be used.

The examples which follow are intended to illustrate the present invention.

### EXAMPLES

### Example 1: The Inflammatory Response can be Induced In Vitro by ICs Containing HIV-1 Transcriptional Transactivator (Tat), which is an Ag with the Ability to Bind Nucleic Acids

### Materials and Methods:

Tat is a protein of 101 residues which was prepared by peptide synthesis as described in the publication by Kittiworakarn et al. (Kittiworakarn et al., 2006). aTat12S and aTat7S antibodies are derived from Tat specific hybridomas; they were obtained following the protocol described in the publication by Lecoq et al. (Lecoq et al., 2008).

The epitopic specificity of these two antibodies was assessed by ELISA. For this purpose, microtitration plates were adsorbed in an amount of 100 μL per well with 10 μg/mL of a peptide having the sequence 1-37 of Tat, called Tat1-37, or with 10 μg/ml of a biotinylated peptide having the sequence 37-57 of Tat, called Tat-37-57-b. The wells were then saturated with 200 μL of bovine serum albumin (BSA) at 0.3%. The plates were then washed and serial dilutions of the two Abs were added. After 4 hours of incubation at room temperature, the plates were washed and a goat anti-mouse antibody coupled to peroxidase was added. After 30 minutes of incubation at room temperature, the plates were washed and ABTS was added. After 30 minutes, the optical density was then measured at 414 nm using an ELISA reader.

To assess the inflammatory ability we first incubated Tat (100 nM final) in the presence or absence of one of the aTat12S and aTat7S antibodies (100 nM final for each Ab), respectively. We then transferred these mixtures into plates containing human peripheral blood mononuclear cells (PBMC) in an amount of 0.1 M cells per well. After 18 h of incubation at 37° C., we collected the supernatants and then assessed whether they contained the pro-inflammatory cytokine IL-6 using an ELISA kit (ref. DY206, RandD systems).

### Results

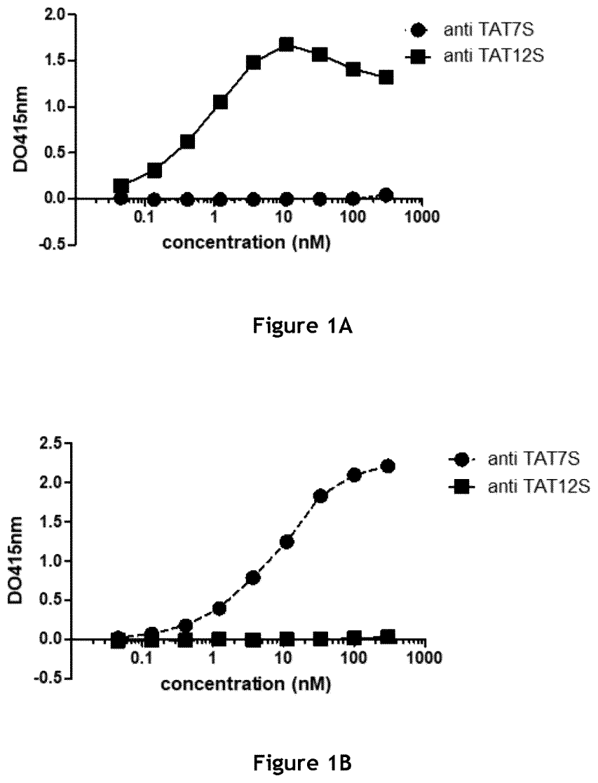

To assess the inflammatory capacity of immune complexes containing an Ag_{nuc}, We first looked at the transcriptional transactivator of HIV-1, called Tat, because this 101 residue long protein has the ability to bind nucleic acids. We used two mouse monoclonal antibodies specific for Tat. These two antibodies are called aTat12S and aTat7S. We first assessed the epitopic specificity of these antibodies by enzyme-linked immunosorbent assay using ELISA plates adsorbed with the peptides Tat1-37 and Tat-37-57-b, respectively. As can be seen in FIG. 1A, aTat12S Ab binds to Tat1-37 but not Tat-37-57, indicating that it recognizes the N-terminal region of Tat. In return, aTat7S Ab does not bind to Tat1-37 but interacts with Tat-37-57-b, indicating that it recognizes Tat region 37-57 (see FIG. 1B). These data therefore indicate that these two antibodies recognize different and non-overlapping epitopes on the Tat protein.

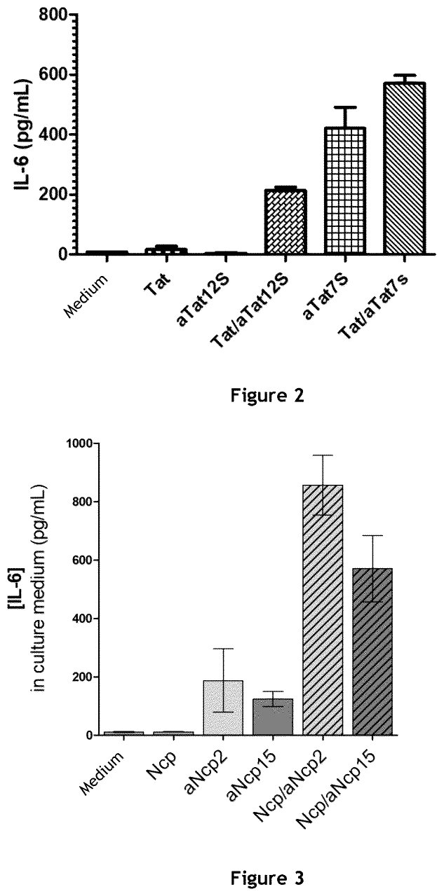

Both aTat12S and aTat7S antibodies are IgG1 isotypes. However, this isotype recognizes the type II Fc gamma receptors, (FcγRII), in humans (Temming et al., 2020). This observation led us to assess whether the ICs Tat/aTat12S and Tat/aTat7S can cause in vitro the secretion of the inflammatory cytokine IL-6 by human PBMCs. To do this, we incubated human PBMCs in the presence of these mixtures, or free Tat, or free antibodies. After 18 h of incubation, we collected the supernatants to assess the presence of IL-6, a cytokine produced during the inflammatory response. We have thus observed that free Tat and aTat12S do not induce IL-6 secretion (see FIG. 2). In return, the Tat/aTat12S IC induces the secretion of IL-6 which indicates that this IC induces an inflammatory response of PBMCs. Free aTat7S Ab induces IL-6 secretion but this secretion is increased when the IC is formed. These data therefore indicate that these IC_{nuc} induce inflammation or amplify it, depending on the Ab included in the complex. Since mouse IgG1 binds in humans only to FcγRIIs, we conclude, moreover, that the inflammatory effect provided by such complexes depends on this type of Fc receptor.

### Example 2: The Inflammatory Response can be Induced by ICs Containing the Nucleocapsid Protein (Ncp) of SARS-COV-2 Virus, which is an Ag with the Ability to Bind Nucleic Acids

### Materials and Methods

### Preparation of SARS-COV-2 Nucleocapsid (Ncp) Protein

The nucleocapsid protein of SARS-COV-2 virus, called Ncp, has the sequence having the code GenBank YP_009724397.2, GenBank version 241 of 15 Dec. 2020. It was prepared recombinantly using a protocol similar to that described in the publication by Stadlbauer et al. (Stadlbauer et al., 2020). Thus, the sequence coding for Ncp was inserted into the plasmid pcDNA3.4. HEK cells (2.5.10⁶ cells/mL) were incubated with plasmid DNA (1 μg/μL) in FreeStyle 293F medium, then with PEI at 0.5 mg/mL. Finally, they were incubated at 37° C. After 2 days, the cells were diluted to a half in FreeStyle 293F medium. On the fifth day, the cells were centrifuged. The supernatants recovered were then passed through a HiTrap Heparin column (GE ref 71-7004-01). The protein retained on the column, corresponding to Ncp, was then eluted in 1.5 M sodium phosphate buffer pH 7 and then concentrated on Vivaspin MWCO=10000 with a 100 mm sodium phosphate buffer pH 7 +150 mm NaCl pH 7. It was stored at −20° C. until use.

### Production of Anti-Ncp Monoclonal Antibodies

The anti-Ncp monoclonal antibodies were produced using a protocol similar to the one described in the publication by Féraudet Tarisse et al. (Tarisse et al., 2021). Biozzi mice were immunized four times at three week intervals by injection of 10 μg Ncp with aluminium hydroxide adjuvant gel, followed by three injections of 50 μg Ncp at one day intervals. Two mice were selected for the production of monoclonal antibodies according to the method developed by (Köhler & Milstein, 1975). The monoclonal antibodies produced by culture supernatant were purified by protein G affinity chromatography

### Assessment of Inflammatory Ability

To assess the inflammatory ability we first incubated Ncp (30 nM final) in the presence or absence of one of the aNcp2 and anti-Ncp15 antibodies (30 nM final for each Ab), respectively. We then transferred these mixtures into plates containing human peripheral blood mononuclear cells (PBMC) in an amount of 0.1 M cells per well. After 18 h of incubation at 37° C., we collected the supernatants and then assessed whether they contained the pro-inflammatory cytokine IL-6.

### Results

To assess the inflammatory capacity of immune complexes containing an Ag_{nuc}, we addressed the nucleocapsid protein of SARS-COV-2 virus, called Ncp, because this protein has the ability to bind RNA. We used two mouse monoclonal antibodies specific for Ncp. These two antibodies are called aNcp2 and aNcp15. They are IgG1 isotypes and can therefore interact in humans with Fc gamma receptors type II (FcγRII). We pre-incubated Ncp with these two ACs independently to form two ICs. Human PBMCs were then incubated in vitro in the presence of these mixtures, or free Ncp, or free antibodies. After 18 h of incubation, we collected the supernatants to assess the presence of IL-6, a cytokine produced during the inflammatory response. We could thus observe that free Ncp or free antibodies induce a low secretion of IL-6, but that the concentration of this cytokine is significantly increased by the two ICs (see FIG. 3). These data therefore indicate that these IC_{nuc} have an inflammatory capacity. Since mouse IgG1 binds only to FcγRIIs in humans (Temming et al., 2020), we conclude, moreover, that the inflammatory effect of such complexes depends on this type of Fc receptor.

### Example 3: The Inflammatory Response Induced by IC_{nuc} Depends on the Fc Region of the Ab Included in the IC

### Materials and Methods

### Obtaining F(Ab)′2 Fragments by Controlled Proteolysis

A controlled proteolysis of aTat12S and aNcp2 antibodies is carried out to obtain their F(ab)′2. For this, aTat12S and aNcp2 are incubated respectively in the presence of pepsin in pH 3 buffer for one hour at 37° C. The antibodies are then purified by immunoaffinity either on a column containing Tat or on a column containing Ncp. Tat and Ncp are covalently coupled via their amine groups to a pre-activated Sepharose resin of the ‘CNBr activated SepharoseR 4B’ type (Ref: GE17-0430-01; Sigma-Aldrich). The preparation of the column and the implementation of affinity chromatography are done according to the manufacturer's recommendations.

To assess the inflammatory ability of an IC containing F(ab)′2-aNcp2, Ncp is first incubated in the presence or absence of aNcp2 Ab or F(ab)′2-aNcp2 fragment. These mixtures are then transferred into plates containing human peripheral blood mononuclear cells (PBMC) in an amount of 0.1 M cells per well. After 18 h of incubation at 37° C., the supernatants are collected and then it is assessed whether they contain the pro-inflammatory cytokine IL-6.

To assess the inflammatory ability of an IC containing F(ab)′2-aTat12S, the Tat protein is first incubated in the presence or absence of aNcp2 Ab or F(ab)′2-aNcp2 fragment. These mixtures are then transferred into plates containing human peripheral blood mononuclear cells (PBMC) in an amount of 0.1 M cells per well. After 18 h of incubation at 37° C., the supernatants are collected and then it is assessed whether they contain the pro-inflammatory cytokine IL-6.

Cloning of aTat12S and aNcp2 Ab by Molecular Biology and Recombinant Production of Fab Fragments Derived Therefrom.

The hybridomas expressing the aTat12S and aNcp2 antibodies, respectively, are cultured and then used as a source of messenger RNA. From these hybridomas, the sequences of the antibodies are obtained by following the methods described by Stravinskiene (2020) and Meyer et al. 2019. Briefly, hybridoma RNAs (3-10×10⁶ cells) are obtained using the GeneJET RNA Purification Kit (Thermo Fisher Scientific, K0731) and directly used for cDNA synthesis. For each antibody, three reactions are prepared using the SMARTScribe Reverse Transcriptase enzyme (Clontech, 639537), the template-switch oligo universal forward primer (AAGCAGTGGTATCAACGAGTACATrGrG, SEQ ID NO: 1, where rG means that it is a G base of RNA, while the other bases are DNA bases) and one of the following specific primers: TTGTCGTTCACTGCCATCAATC (SEQ ID NO: 2, reverse primer for kappa chain mIGK for RT), GGGGTACCATCTACCTTCCAG (SEQ ID NO: 3, reverse primer for lambda chain mIGL for RT) or AGCTGGAAGGTGTGCACAC (SEQ ID NO: 4, reverse primer for heavy chain mIGHG for RT). The cDNAs are amplified by PCR using the ISPCR universal forward primer (AAGCAGTGGTATCAACGCAGAG, SEQ ID NO: 5) and one of the specific primers: reverse primer for kappa mIGK chain for PCR (ACATTGATGTCTTTGGGGTAGAAG, SEQ ID NO: 6), reverse primer for lambda mIGL chain for PCR (ATCGTACACCAGTGGC, SEQ ID NO: 7) or reverse primer for mIGHG heavy chain for PCR (GGGGATCCAGAGTTCCAGTC, SEQ ID NO: 8).

The amplicons are then analyzed by agarose gel electrophoresis, purified and cloned into plasmid pJet1.2 (Thermo Fisher Scientific, K1231) and sequenced by the Sanger method. From the sequences obtained, new specific primers are used for cloning the VH region of aTat12S or aNcp2 in the vector AbVec-hIgG1 (comprising a human heavy chain constant region of IgG1 isotype, GenBank accession number: FJ475055.1; between the AgeI and ApaI sites, GenBank version 241 of 15 Dec. 2020). Similarly, new specific primers are used for cloning the VL region of aTat12S or aNcp2 in the vector AbVec-hIgKappa (comprising a human light chain constant region of kappa isotype, GenBank accession number: FJ475056.1, between the AgeI and BsiWI sites, GenBank version 241 of 15 Dec. 2020) or AbVec-hIgLambda (comprising a human light chain constant region of lambda isotype, GenBank accession number: FJ517647.1, between the AgeI and XhoI sites, GenBank version 241 of 15 Dec. 2020). These vectors make it possible to produce antibodies directed against chimeric Tat12S and aNcp2, with a human constant region (denoted hcaTat12S and hcaNcp2, where ‘hc’ means that it is a chimeric antibody with a human constant region; this terminology is used in all the examples). The mutations E233P, F234V, L235A and D265A are incorporated into the plasmid AbVec-hIgG1 to obtain variants of the aTat12S and aNcp2 antibodies lacking effector activity. A variant of the plasmid AbVec-hIgG1 making it possible to stop translation after the CH1 domain (stop codon after the C220 position in the sequence K218-S219-C220) is used to obtain two plasmids coding for the VH-CH1 regions of hcaTat12S and hcaNcp2, respectively.

For the expression of recombinant Fab fragments and whole antibodies lacking FcγR binding capacity, 293-F cells (HEK, Thermo Fisher Scientific, R790-07) are co-transfected (equimolar ratio) with a plasmid corresponding either to the VH-CH1 region or to the complete heavy chain of an IgG1 antibody and to the light chain of the kappa or lambda type by the PEI method (0.5 mg/ml; Longo et al. (2013) and cultured for 5-8 days in FreeStyle™ 293 Expression Medium (Thermo Fisher Scientific, 12338-018). The Fabs labeled with their poly-histidine motifs in the medium are purified by immobilized metal ion affinity chromatography (nickel column coupled to Fast Flow Sepharose beads (GE Healthcare, 17-0575-01)-3). The whole antibodies secreted into the medium are purified by protein A affinity chromatography (Millipore, 113115827).

For expression of whole antibodies lacking FcγR binding capacity, 293-F cells (HEK, Thermo Fisher Scientific, R790-07) are co-transfected (equimolar ratio) with a plasmid corresponding to the heavy chain of an IgG1 antibody and either a light chain of the kappa or lambda type by the PEI method (0.5 mg/ml; Longo et al. 2013) and cultured for 5-8 days in FreeStyle™ 293 Expression Medium (Thermo Fisher Scientific, 12338-018). The antibodies secreted into the medium are purified by protein A affinity chromatography (Millipore, 113115827).

### Results

A) Study of the Inflammatory Effect with F(Ab)′2 Fragments Obtained by Controlled Proteolysis

Since ICs generally interact, via the Fc domain of antibodies, with FcγRs, it is assessed whether the Fc domain of an antibody included in an IC_{nuc} is involved in the inflammatory response. For this purpose, the aTat12S Ab and aNcp2 Ab are used. Here, it is chosen to alter their ability to bind FcγRs by eliminating their respective Fc domains.

These domains are eliminated by proteolysis with pepsin. This controlled proteolysis makes it possible to obtain F(ab)′2 fragments, respectively called F(ab)′2-aTat12S and F(ab)′2-aNcp2. These two Ab fragments lacking the Fc region are then incubated with their respective Ag_{nuc}. Two ICs, respectively called IC-F(ab)′2-aTat12S and IC-F(ab)′2-aNcp2, are thus formed. They are then compared to ICs containing whole antibodies, respectively called IC-Ab-aTat12S and IC-Ab-aNcp2, for the ability to induce IL-6 secretion by human PBMCs.

Under these experimental conditions, it is expected that IC-Ab-aTat12S and IC-Ab-aNcp2 behave as in Examples 1 and 2, i.e., they trigger IL-6 secretion by PBMCs. In return, it is expected that IC-F(ab)′2-aTat12S and IC-F(ab)′2-aNcp2 exhibit reduced or no inflammatory capacity, due to the absence of Fc region that obliterates the ability to bind FcγRs.

B) Study of the Inflammatory Effect with the Fab Fragments Obtained by the Recombinant Route

Since ICs generally interact, via the Fc domain of antibodies, with FcγRs, it is assessed whether the Fc domain of an antibody included in an IC_{nuc} is involved in the inflammatory response. For this purpose, the aTat12S Ab and aNcp2 Ab are used. Here, it is chosen to alter their ability to bind FcγRs from recombinant constructs lacking their respective Fc domains. By molecular biology, Fab fragments, respectively called Fab-aTat12S and Fab-aNcp2, are produced. These two Ab fragments lacking the Fc region are then incubated with their respective Ag_{nuc}. Two ICs, respectively called IC-Fab-aTat12S and IC-Fab-aNcp2, are thus formed. They are then compared to ICs containing whole antibodies, respectively called IC-Ab-aTat12S and IC-Ab-aNcp2, for the ability to induce IL-6 secretion by human PBMCs.

Under these experimental conditions, it is expected that IC-Ab-aTat12S and IC-Ab-aNcp2 behave as in Examples 1 and 2, i.e., they trigger IL-6 secretion by PBMCs. In return, it is expected that IC-Fab-aTat12S and IC-Fab-aNcp2 exhibit reduced or no inflammatory capacity, due to the absence of Fc region that obliterates the ability to bind FcγRs.

C) Study of the Inflammatory Effect with aTat12S and aNcp2 Antibodies Lacking the Ability to Bind Fcγ Receptors.

Since ICs generally interact, via the Fc domain of antibodies, with FcγRs, it is assessed whether the Fc domain of an antibody included in an IC_{nuc} is involved in the inflammatory response. For this purpose, the aTat12S Ab and aNcp2 Ab are used. Here, it is chosen to alter their ability to bind FcγRs from recombinant constructs mutated in their respective Fc domains. By molecular biology, these mutated antibodies, respectively designated hcaTat12S-FCₘᵤₜ and hcaNcp2-FCₘᵤₜ, are produced. These two mutated antibodies are then incubated with their respective Ag_{nuc}. It is thus possible to form two ICs, respectively called IC-hcaTat12S-FCₘᵤₜ and IC-hcaNcp2-FCₘᵤₜ. They are then compared to ICs containing unmutated antibodies, respectively called IC-Ab-aTat12S and IC-Ab-aNcp2, for the ability to induce IL-6 secretion by human PBMCs.

Under these experimental conditions, it is expected that IC-Ab-aTat12S and IC-Ab-aNcp2 behave as in Examples 1 and 2, i.e., they trigger IL-6 secretion by PBMCs. In return, it is expected that IC-hcaTat12S-FCₘᵤₜ and IC-hcaNcp2-FCₘᵤₜ exhibit reduced or no inflammatory capacity, due to the absence of Fc region that obliterates the ability to bind FcγRs.

### Example 4: The Inflammatory Reaction Induced by Whole Antibodies is Blocked by an Ab Derivative Impaired in its Ability to Bind FcγRs

### Materials and Methods

### Assessment of Inflammation Blockade by F(Ab)′2

To assess whether F(ab)′2-aNcp2 can block inflammation caused by an IC_{nuc} containing Ncp, Ncp is first incubated alone or with aNcp2 and in the presence or absence of F(ab)′2-aNcp2. To assess whether an anti-inflammatory effect can be provided by non-specific F(ab)′2, Ncp is also incubated with aNcp2 Ab in the presence or absence of F(ab)′2-aTat12S.

To assess whether F(ab)′2-anti-Tat can block inflammation caused by an IC_{nuc} containing Tat, the Tat protein is first incubated alone or with aTat12S Ab and in the presence or absence of F(ab)′2-aTat12S. To assess whether the anti-inflammatory effect can be provided by non-specific F(ab)′2, Tat is also incubated with aTat12S Ab in the presence or absence of F(ab)′2-aNcp2.

These various mixtures are then transferred into plates containing human peripheral blood mononuclear cells (PBMC) in an amount of 0.1 M cells per well. After 18 h of incubation at 37° C., the supernatants are collected and then it is assessed whether they contain the pro-inflammatory cytokine IL-6.

### Assessment of Inflammation Blockade by Fab

To assess whether Fab-aNcp2 can block inflammation caused by an IC_{nuc} containing Ncp, Ncp is first incubated alone or with aNcp2 Ab and in the presence or absence of F(ab)′2-aNcp2. To assess whether the anti-inflammatory effect can be provided by a non-specific Fab2, Ncp is also incubated with aNcp2 Ab in the presence or absence of Fab-aTat12S.

To assess whether Fab-anti-Tat can block inflammation caused by an IC_{nuc} containing Tat, the Tat protein is first incubated alone with aTat12S Ab and in the presence or absence of Fab-aTat12S. To assess whether the anti-inflammatory effect can be provided by a non-specific Fab, Tat is also incubated with aTat12S Ab in the presence or absence of Fab-aNcp2.

These various mixtures are then transferred into plates containing human peripheral blood mononuclear cells (PBMC) in an amount of 0.1 M cells per well. After 18 h of incubation at 37° C., the supernatants are collected and then it is assessed whether they contain the pro-inflammatory cytokine IL-6.

Assessment of Inflammation Blockade by Whole hcaTat12S and hcaNcp2 Antibodies Lacking the Ability to Bind Fcγ Receptors.

To assess whether an Ab lacking FcγR-binding capacity can block inflammation caused by an IC_{nuc} containing Ncp, Ncp is first incubated alone or with aNcp2 Ab and in the presence or absence of hcaNcp2-FCₘᵤₜ Ab. To assess whether the anti-inflammatory effect can be provided by non-specific F(ab)′2, Ncp is also incubated with aNcp2 Ab in the presence or absence of hcaTat12S-FCₘᵤₜ.

To assess whether an Ab lacking FcγR-binding capacity can block inflammation caused by an IC_{nuc} containing Tat, Tat is first incubated alone or with aTat12S Ab and in the presence or absence of hcaTat12S-FCₘᵤₜ. To assess whether the anti-inflammatory effect can be provided by a non-specific F(ab)′2, Tat is incubated with aTat12S Ab in the presence or absence of hcaNcp2-FCₘᵤₜ Ab.

These various mixtures are then transferred into plates containing human peripheral blood mononuclear cells (PBMC) in an amount of 0.1 M cells per well. After 18 h of incubation at 37° C., the supernatants are collected and then it is assessed whether they contain the pro-inflammatory cytokine IL-6.

### Results

### Assessment of Inflammation Blockade by F(Ab)′2

To assess whether inflammation induced by ICs containing Tat and aTat12S Ab or aTat7S Ab can be reduced or even abolished in the presence of an anti-Tat Ab lacking FcγR binding capacity, the F(ab)′2-aTat12S described in Example 3 is used. Tat is incubated with one of the two whole antibodies in the absence or presence of F(ab)′2-aTat12S to form different types of immune complexes. These mixtures are then incubated for 24 hours with PBMCs and the presence of IL-6 in the supernatants is measured.

Under these experimental conditions, it is expected that IC-Ab-aTat12S behaves as in Example 1, i.e., it triggers IL-6 secretion by PBMCs. It is also expected that this secretion will not be significantly altered when Tat, aTat12S and F(ab)′2-aNcp2 are incubated individually. In return, it is expected that this secretion will be reduced or zero when Tat, aTat12S and F(ab)′2-aTat12S are incubated together.

To assess whether inflammation induced by ICs containing Ncp and aNcp2 Ab or aNcp15 Ab can be reduced or even abolished in the presence of an anti-Ncp Ab lacking FcγR binding capacity, the F(ab)′2-aNcp2 described in Example 3 is used. Ncp is incubated with one of the two whole antibodies in the absence or presence of F(ab)′2-aNcp2 to form different types of immune complexes. These mixtures are then incubated for 24 hours with PBMCs and the presence of IL-6 in the supernatants is measured.

Under these experimental conditions, it is expected that IC-Ab-aNcp2 behaves as in Example 2, i.e., it triggers IL-6 secretion by PBMCs. It is also expected that this secretion will not be significantly altered when Ncp, aNcp2 and F(ab)′2-aTat12S are incubated individually. In return, it is expected that this secretion will be reduced or zero when Ncp, aNcp2 and F(ab)′2-aNcp2 are incubated together.

### Assessment of Inflammation Blockade by Fab

To assess whether inflammation induced by ICs containing Tat and aTat12S Ab or aTat7S Ab can be reduced or even abolished in the presence of an anti-Tat Ab lacking FcγR binding capacity, the Fab-aTat12S described in Example 3 is used. Tat is incubated with one of the two whole antibodies in the absence or presence of Fab-aTat12S to form different types of immune complexes. These mixtures are then incubated for 24 hours with PBMCs and the presence of IL-6 in the supernatants is measured.

Under these experimental conditions, it is expected that IC-Ab-aTat12S behaves as in Example 1, i.e., it triggers IL-6 secretion by PBMCs. It is also expected that this secretion will not be significantly altered when Tat, aTat12S and Fab-aNcp2 are incubated individually. In return, it is expected that this secretion will be reduced or zero when Tat, aTat12S and Fab-aTat12S are incubated together.

To assess whether inflammation induced by ICs containing Ncp and aNcp2 Ab or aNcp15 Ab can be reduced or even abolished in the presence of an anti-Ncp Ab lacking FcγR binding capacity, the Fab-aNcp2 described in Example 3 is used. Ncp is incubated with one of the two whole antibodies in the absence or presence of Fab-aNcp2 to form different types of immune complexes. These mixtures are incubated for 24 hours with PBMCs and the presence of IL-6 in the supernatants is measured.

Under these experimental conditions, it is expected that IC-Ab-aNcp2 behaves as in Example 2, i.e., it triggers IL-6 secretion by PBMCs. It is also expected that this secretion will not be significantly altered when Ncp, aNcp2 and Fab-aTat12S are incubated individually. In return, it is expected that this secretion will be reduced or zero when Ncp, aNcp2 and Fab-aNcp2 are incubated together.

Assessment of Inflammation Blockade with Whole hcaTat12S and hcaNcp2 Antibodies Lacking the Ability to Bind Fc Receptors.

To assess whether inflammation induced by ICs containing Tat and aTat12S Ab or aTat7S Ab can be reduced or even abolished in the presence of an anti-Tat Ab lacking FcγR binding capacity, the mutated hcaTat12S antibody (hcaTat12S-FCₘᵤₜ) described in Example 3 is used. Tat is incubated with one of the two whole antibodies in the absence or presence of hcaTat12S-FCₘᵤₜ to form different types of immune complexes. These mixtures are then incubated for 24 hours with PBMCs and the presence of IL-6 in the supernatants is measured.

Under these experimental conditions, it is expected that IC-Ab-hcaTat12S behaves as in Example 1, i.e., it triggers IL-6 secretion by PBMCs. It is also expected that this secretion will not be significantly altered when Tat, aTat12S and hcaNcp2-FCₘᵤₜ are incubated individually. In return, it is expected that this secretion will be reduced or zero when Tat, aTat12S and hcaNcp2-FCₘᵤₜ are incubated together.

To assess whether inflammation induced by ICs containing Ncp and aNcp2 Ab or aNcp15 Ab can be reduced or even abolished in the presence of an anti-Ncp Ab lacking FcγR binding capacity, the mutated hcaNcp2 antibody (hcaNcp2 FCₘᵤₜ) described in Example 3 is used. Ncp is incubated with one of the two whole antibodies in the absence or presence of hcaNcp2 Fcₘᵤₜ to form different types of immune complexes. These mixtures are then incubated for 24 hours with PBMCs and the presence of IL-6 in the supernatants is measured.

Under these experimental conditions, it is expected that IC-Ab-aNcp2 behaves as in Example 2, i.e., it triggers IL-6 secretion by PBMCs. It is also expected that this secretion will not be significantly altered when Ncp, aNcp2 and hcaNcp2 FCₘᵤₜ are incubated individually. In return, it is expected that this secretion will be reduced or zero when Ncp, aNcp2 and hcaNcp2-FCₘᵤₜ are incubated together.

### Example 5: The Inflammatory Response Induced by IC_{nuc} Depends on the Fc Region of the Ab Included in the IC

### Materials and Methods

Cloning of aNcp15 Ab by Molecular Biology and Recombinant Production of Fab Fragments and Antibodies Lacking the Ability to Bind FcγRs Derived Therefrom.

The hybridoma expressing the aNcp15 Ab antibody is cultured and then used as a source of messenger RNA. From this hybridoma, the sequences of the antibodies are obtained by following the methods described by Stravinskiene (2020) and Meyer et al. 2019. Briefly, hybridoma RNAs (3-10×10⁶ cells) are obtained using the GeneJET RNA Purification Kit (Thermo Fisher Scientific, K0731) and directly used for cDNA synthesis. For each antibody, three reactions are prepared using the SMARTScribe Reverse Transcriptase enzyme (Clontech, 639537), the template-switch oligo universal forward primer (AAGCAGTGGTATCAACGAGTACATrGrG, SEQ ID NO: 1, where rG means that it is a G base of RNA, while the other bases are DNA bases) and one of the following specific primers: TTGTCGTTCACTGCCATCAATC (SEQ ID NO: 2, reverse primer for kappa chain mIGK for RT), GGGGTACCATCTACCTTCCAG (SEQ ID NO: 3, reverse primer for lambda chain mIGL for RT) or AGCTGGAAGGTGTGCACAC (SEQ ID NO: 4, reverse primer for heavy chain mIGHG for RT). The cDNAs are amplified by PCR using the ISPCR universal forward primer (AAGCAGTGGTATCAACGCAGAG, SEQ ID NO: 5) and one of the specific primers: reverse primer for kappa mIGK chain for PCR (ACATTGATGTCTTTGGGGTAGAAG, SEQ ID NO: 6), reverse primer for lambda mIGL chain for PCR (ATCGTACACCAGTGGC, SEQ ID NO: 7) or reverse primer for mIGHG heavy chain for PCR (GGGGATCCAGAGTTCCAGTC, SEQ ID NO: 8).

The amplicons are then analyzed by agarose gel electrophoresis, purified and cloned into plasmid pJet1.2 (Thermo Fisher Scientific, K1231) and sequenced by the Sanger method. From the sequences obtained, new specific primers are used for cloning the VH region of aNcp15 in the vector AbVec-hIgG1 (comprising a human heavy chain constant region of IgG1 isotype, GenBank accession number: FJ475055.1; between the AgeI and ApaI sites, GenBank version 241 of 15 Dec. 2020). Similarly, new specific primers are used for cloning the VL region of aNcp15 in the vector AbVec-hIgKappa (comprising a human light chain constant region of kappa isotype, GenBank accession number: FJ475056.1, between the AgeI and BsiWI sites, GenBank version 241 of 15 Dec. 2020) or AbVec-hIgLambda (comprising a human light chain constant region of lambda isotype, GenBank accession number: FJ517647.1, between the AgeI and XhoI sites, GenBank version 241 of 15 Dec. 2020). These vectors encode the heavy and light chains of a chimeric antibody with human constant regions denoted hcaNcp15 (where ‘hc’ indicates that the antibody is chimeric with human constant regions; this terminology is used in all examples). The mutations E233P, F234V, L235A and D265A are incorporated into the plasmid AbVec-hIgG1 to obtain variants of the aNcp15 antibodies lacking effector activity. A plasmid derived from AbVec-hIgG1 was constructed by the insertion of the sequence coding for ENLYFQSHHHHH (cleavage site by TEV protease followed by 6 histidines) downstream of cysteine 220 (C220, in the sequence K218-S219-C220) followed by a stop codon allowing translation to be stopped. This plasmid (AbVec-hIgG1_Fab-TEV-6×His) is used for the expression of the VH-CH1-TEV-6×His fusion of aNcp15 necessary for the production of the corresponding Fab (Fab-aNcp15).

For the expression of recombinant Fab fragments and whole antibodies lacking FcγR binding capacity, 293-F cells (HEK, Thermo Fisher Scientific, R790-07) are co-transfected (equimolar ratio) with a plasmid corresponding either to the VH-CH1 region or to the complete heavy chain of an IgG1 antibody and the light chain of the kappa or lambda type by the PEI method (0.5 mg/mL; Longo et al. (2013) and cultured for 5-8 days in FreeStyle™ 293 Expression Medium (Thermo Fisher Scientific, 12338-018). Fabs containing poly-histidine labels and secreted into the medium are purified by IMAC (immobilized metal ion affinity chromatography, GE Healthcare Cat.N. 17-0575-01). The whole antibodies secreted into the medium are purified by protein A affinity chromatography (Millipore, 113115827).

For expression of whole antibodies lacking FcγR binding capacity, 293-F cells (HEK, Thermo Fisher Scientific, R790-07) are co-transfected (equimolar ratio) with a plasmid corresponding to the heavy chain of an IgG1 antibody and either a light chain of the kappa or lambda type by the PEI method (0.5 mg/ml; Longo et al. 2013) and cultured for 5-8 days in FreeStyle™ 293 Expression Medium (Thermo Fisher Scientific, 12338-018). The antibodies secreted into the medium are purified by protein A affinity chromatography (Millipore, 113115827).

Immunoenzymatic Study of hcaNcp15, Mutated hcaNcp15Fc and Fab-aNcp15 for the Ability to Bind the Ncp Protein.

For this study the antibody hcaNcp15 was adsorbed onto microtiter plates (0.1 μg/100 μL/well). The plates were then saturated with a PBS buffer containing 0.3% bovine serum albumin. The plates were then washed and a fixed concentration of N-biotinylated protein (178 pM) was added into the wells in the presence or absence of hcaNcp15 or mutated hcaNcp15c or Fab-aNcp15 serial dilutions. After overnight incubation at 4° C., the plate was washed and streptavidin coupled to peroxidase was added. After 30 minutes, washings were carried out and a colorimetric substrate (ABTS) was added to measure the presence of biotinylated Ncp.

### Results

A) Recombinant Production of the Mutated aNcp15Fc Ab and the Fab-aNcp15 Fragment, and Characterization of their Ability to Bind the Ncp Protein.

Since ICs generally interact, via the Fc domain of antibodies, with FcγRs, it is assessed whether the Fc domain of an antibody included in an IC_{nuc} is involved in the inflammatory response. For this, the hcaNcp15 Ab is used. Here, it is chosen to produce two recombinant constructs lacking the capacity to bind FcγRs. The first construct corresponds to the Fab fragment of aNcp15, called Fab-aNcp15. The second, corresponds to the hcaNcp15 Ab containing a mutated Fc domain in order to impair its ability to bind FcγRs. This construct is called mutated hcaNcp15Fc.

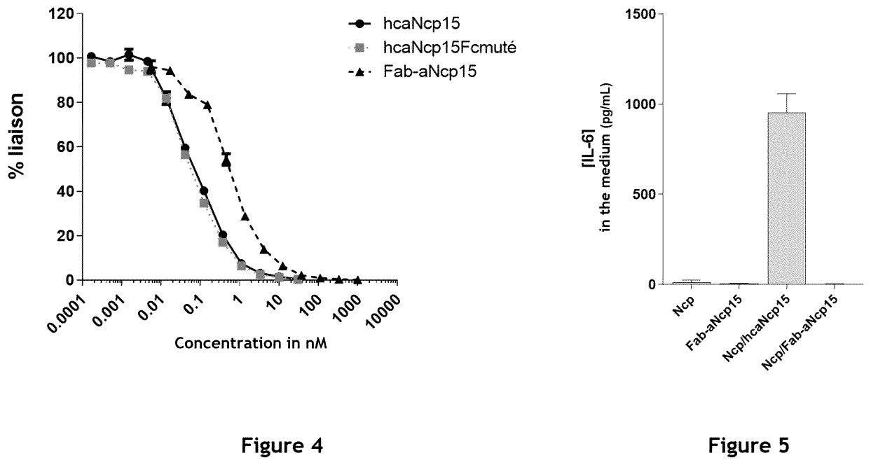

The ability of Fab-aNcp15 and mutated hcaNcp15Fc to bind Ncp is then compared to that of the whole hcaNcp15 Ab by enzyme immunoassay. For this purpose, serial dilutions of Fab-aNcp15, mutated hcaNcp15Fc or hcaNcp15 Ab were incubated in the presence of a fixed amount of biotinylated Ncp in plates previously adsorbed with hcaNcp15 Ab. The plates were then washed and incubated in the presence of streptavidin-peroxidase and a colorimetric substrate to assess the binding of Ncp-biotin to the microtitration wells. As can be seen in FIG. 4, binding of Ncp-biotin to the plates is inhibited in a dose-dependent manner by the whole hcaNcp15 Ab, by mutated hcaNcp15Fc and by Fab-aNcp15. These data therefore indicate that mutated hcaNcp15Fc and the Fab fragment retain the ability to interact with the Ncp protein. The whole hcaNcp15 Ab and mutated hcaNcp15Fc Ab have similar inhibitory capacity, indicating that mutations in the Fc domain of the Ab do not alter binding capacity. The whole Ab, on the other hand, has a higher inhibitory capacity than the Fab fragment, which is expected because the integral molecule has two Fab domains that contribute to the avidity of the interaction.

B) Study of the Inflammatory Effect with the Fab Fragment Obtained by the Recombinant Route

To assess whether the inflammatory effect mediated by an immune complex depends on the Fc domain of the Ab, the previously produced Fab-aNcp15 fragment is used. It is incubated in the presence of the Ncp protein to form an IC called Ncp/Fab-aNcp15. Then its ability to induce IL-6 secretion by human PBMCs is compared with that of free Ncp, free Fab-aNcp15, and the Ncp/aNcp15 IC. The latter IC, whose ability to trigger IL-6 secretion is shown in FIG. 3, is used as a positive control.

Under these experimental conditions, the free Fab-aNcp15, the free Ncp protein and the IC containing Fab-aNcp15 behave in the same way since they exhibit almost no inflammatory capacity (FIG. 5). The IC aNcp15 used as a positive control triggers the secretion of IL-6 by PBMCs. This last observation indicates that the absence of effect of the Ncp/Fab-aNcp15 is not due to a loss of cell reactivity. These results therefore demonstrate that IL-6 secretion cannot be caused by an immune complex containing a Fab. They also indicate that the inflammatory effect depends on the presence of the Fc region in the complex.

C) Study of the Inflammatory Effect with hcaNcp15 Ab Lacking the Ability to Bind Fcγ Receptors.

To assess whether the inflammatory effect mediated by an immune complex depends on the ability to bind RFcgs, the mutated hcaNcp15Fc Ab lacking the ability to bind Fcγ receptors is used. This mutated Ab is incubated with Ncp so as to form an immune complex called Ncp/hcaNcp15-FCₘᵤₜ. This complex is compared with i) the IC containing the unmutated whole Ab called Ncp/hcaNcp15, ii) hcaNcp15-FCₘᵤₜ, iii) free Ncp for the ability to induce IL-6 secretion by human PBMCs.

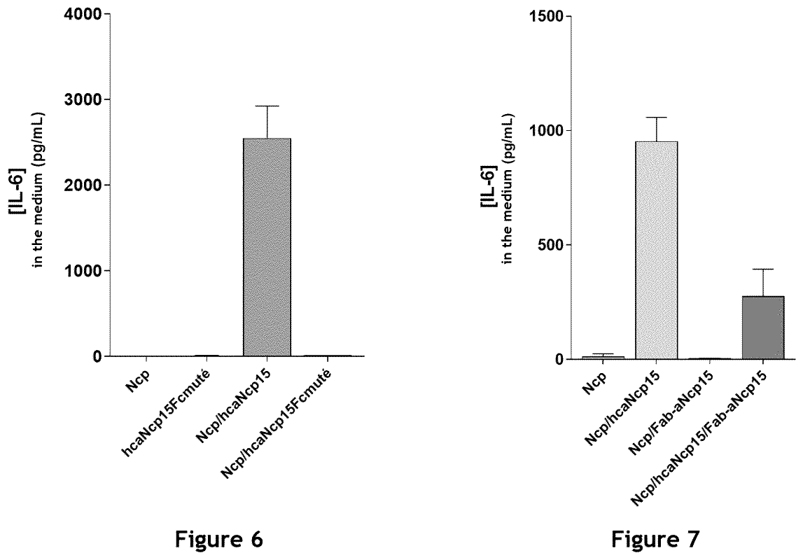

Under these experimental conditions, the Ncp/hcaNcp15-FCₘᵤₜ IC, the free Ncp protein and hcaNcp15-Fcₘᵤₜ behave in the same way since they exhibit almost no inflammatory capacity (FIG. 6). In return, the Ncp/hcaNcp15 IC behaves as in Examples 1 and 2, i.e., it triggers the secretion of IL-6 by PBMCs. These results indicate that the secretion of IL-6 cannot be induced with an Ab whose Fc region is impaired for the ability to bind FcγRs, which demonstrates that the phenomenon depends on this ability to interact.

### Example 6: The Inflammatory Reaction Induced by Whole Antibodies is Blocked by an Ncp-Specific Fab or by an Ab Derivative Impaired in its Ability to Bind FcγRs

### Materials and Methods

### Assessment of Inflammation Blockade by an Ncp-Specific Fab

To assess whether Fab-aNcp15 can block inflammation caused by an IC_{nuc} containing Ncp, the hcaNcp15 Ab previously described in Example 2 is used. Ncp is incubated alone or with hcaNcp15 and in the presence or absence of Fab-aNcp15. Ncp is also incubated in the presence of Fab-aNcp15 as a control. These various mixtures are then transferred into plates containing human peripheral blood mononuclear cells (PBMC) in an amount of 0.1 M cells per well. After 18 h of incubation at 37° C., the supernatants are collected and then it is assessed whether they contain the pro-inflammatory cytokine IL-6.

Assessment of Inflammation Blockade by Whole aNcp15 Ab Lacking the Ability to Bind Fc Receptors.

To assess whether an Ab lacking FcγR binding capacity can block inflammation caused by an IC_{nuc} containing Ncp, Ncp is first incubated alone or with hcNcp15 Ab and in the presence or absence of mutated hcaNcp15 Ab.

These various mixtures are then transferred into plates containing human peripheral blood mononuclear cells (PBMC) in an amount of 0.1 M cells per well. After 18 h of incubation at 37° C., the supernatants are collected and then it is assessed whether they contain the pro-inflammatory cytokine IL-6.

### Results

### Assessment of Inflammation Blockade by a Fab Fragment

To assess whether inflammation induced by ICs containing Ncp can be impaired in the presence of an Ncp-specific Fab, hcaNcp15 Ab and Fab-aNcp15 are used. Ncp is incubated alone or in the presence of Fab-aNcp15. Ncp is also incubated with whole hcaNcp15 Ab in the absence or presence of Fab-aNcp15 to form different types of immune complexes. These mixtures are then incubated for 18 hours with PBMCs and the presence of IL-6 in the supernatants is measured.

Under these experimental conditions, we observed that the Ncp/hcaNcp15 IC triggers the secretion of IL-6 by PBMCs (see FIG. 7). In return, this secretion is reduced when Ncp, hcaNcp15 and Fab-aNcp2 are incubated together. These results indicate that immune complex-induced IL-6 secretion may be impaired in the presence of an Ncp-specific Fab.

Assessment of Inflammation Blockade by Mutated hcaNcp15Fc Ab Lacking the Ability to Bind Fcγ Receptors.

To assess whether inflammation induced by ICs containing Ncp can be impaired in the presence of mutated antibodies lacking the ability to bind Fcγ receptors, hcaNcp15 Ab and mutated hcaNcp15Fc Ab are used. Ncp is incubated alone or in the presence of mutated hcaNcp15Fc. Ncp is also incubated with whole hcaNcp15 Ab in the absence or presence of mutated hcaNcp15Fc to form different types of immune complexes. These mixtures are then incubated for 18 hours with PBMCs and the presence of IL-6 in the supernatants is measured.

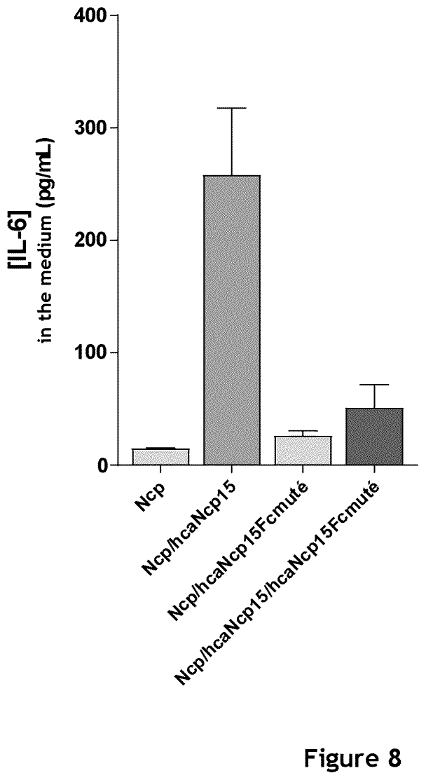

Under these experimental conditions, we observed that the Ncp/hcaNcp15 IC triggers the secretion of IL-6 by PBMCs (see FIG. 8). In return, this secretion is reduced when Ncp, hcaNcp15 and mutated hcaNcp15 are incubated together. These results indicate that the secretion of IL-6 induced by an immune complex can be impaired in the presence of a specific Ab whose Fc region is impaired for the ability to bind FcγRs.

### REFERENCES

Almagro, J. C., & Fransson, J. (2008). Humanization of antibodies. Frontiers in Bioscience: A Journal and Virtual Library, 13, 1619-1633. http://www.ncbi.nlm.nih.gov/pubmed/17981654
Ben Mkaddem, S., Benhamou, M., & Monteiro, R. C. (2019). Understanding Fc Receptor Involvement in Inflammatory Diseases: From Mechanisms to New Therapeutic Tools. Frontiers in Immunology, 10, 811. https://doi.org/10.3389/fimmu.2019.00811
Cafaro, A., Tripiciano, A., Picconi, O., Sgadari, C., Moretti, S., Buttò, S., Monini, P., & Ensoli, B. (2019). Anti-Tat Immunity in HIV-1 Infection: Effects of Naturally Occurring and Vaccine-Induced Antibodies Against Tat on the Course of the Disease. Vaccines, 7(3). https://doi.org/10.3390/vaccines7030099
Carter, J. J., Koutsky, L. A., Hughes, J. P., Lee, S. K., Kuypers, J., Kiviat, N., & Galloway, D. A. (2000). Comparison of human papillomavirus types 16, 18, and 6 capsid antibody responses following incident infection. The Journal of Infectious Diseases, 181(6), 1911-1919. https://doi.org/10.1086/315498
Cecconi, M., Evans, L., Levy, M., & Rhodes, A. (2018). Sepsis and septic shock. Lancet (London, England), 392(10141), 75-87. https://doi.org/10.1016/S0140-6736(18) 30696-2
Chenoweth, A. M., Wines, B. D., Anania, J. C., & Mark Hogarth, P. (2020). Harnessing the immune system via FcγR function in immune therapy: a pathway to next-gen mAbs. Immunology and Cell Biology, 98(4), 287-304. https://doi.org/10.1111/imcb. 12326
Choi, M. Y., FitzPatrick, R. D., Buhler, K., Mahler, M., & Fritzler, M. J. (2020). A review and meta-analysis of anti-ribosomal P autoantibodies in systemic lupus erythematosus. Autoimmunity Reviews, 19(3), 102463. https://doi.org/10.1016/j.autrev.2020.102463
Combes, A. J., Courau, T., Kuhn, N. F., Hu, K. H., Ray, A., Chen, W. S., Chew, N. W., Cleary, S. J., Kushnoor, D., Reeder, G. C., Shen, A., Tsui, J., Hiam-Galvez, K. J., Muñoz-Sandoval, P., Zhu, W. S., Lee, D. S., Sun, Y., You, R., Magnen, M., Krummel, M. F. (2021). Global absence and targeting of protective immune states in severe COVID-19. Nature. https://doi.org/10.1038/s41586-021-03234-7
Dinarello, C. A. (2010). Anti-inflammatory Agents: Present and Future. Cell, 140(6), 935-950. https://doi.org/10.1016/j.cell.2010.02.043
Edelman, G. M., Cunningham, B. A., Gall, W. E., Gottlieb, P. D., Rutishauser, U., & Waxdal, M. J. (1969). The covalent structure of an entire gammaG immunoglobulin molecule. Proceedings of the National Academy of Sciences of the United States of America, 63(1), 78-85. https://doi.org/10.1073/pnas.63.1.78
Fenouillet, E., Blanes, N., Coutellier, A., & Gluckman, J. C. (1993). Relationship between anti-p24 antibody levels and p24 antigenemia in HIV-infected patients. AIDS Research and Human Retroviruses, 9(12), 1251-1255. https://doi.org/10.1089/aid. 1993.9.1251
Fu, Y., Cheng, Y., & Wu, Y. (2020). Understanding SARS-COV-2-Mediated Inflammatory Responses: From Mechanisms to Potential Therapeutic Tools. Virologica Sinica, 35(3), 266-271. https://doi.org/10.1007/s12250-020-00207-4
Gao, T., Hu, M., Zhang, X., Li, H., Zhu, L., Liu, H., Dong, Q., Zhang, Z., Wang, Z., Hu, Y., Fu, Y., Jin, Y., Li, K., Zhao, S., Xiao, Y., Luo, S., Li, L., Zhao, L., Liu, J., . . . Cao, C. (2020). Highly pathogenic coronavirus N protein aggravates lung injury by MASP-2-mediated complement over-activation. MedRxiv, 2020.03.29.20041962. https://doi.org/10.1101/2020.03.29.20041962
Ghiggeri, G. M., D'Alessandro, M., Bartolomeo, D., Degl'Innocenti, M. L., Magnasco, A., Lugani, F., Prunotto, M., & Bruschi, M. (2019). An Update on Antibodies to Necleosome Components as Biomarkers of Sistemic Lupus Erythematosus and of Lupus Flares. International Journal of Molecular Sciences, 20(22). https://doi.org/10.3390/ijms20225799
Hashida, S., Hashinaka, K., Ishikawa, S., & Ishikawa, E. (1997). More reliable diagnosis of infection with human immunodeficiency virus type 1 (HIV-1) by detection of antibody IgGs to pol and gag proteins of HIV-1 and p24 antigen of HIV-1 in urine, saliva, and/or serum with highly sensitive and specific enzyme immunoas. Journal of Clinical Laboratory Analysis, 11(5), 267-286. http://www.ncbi.nlm.nih.gov/pubmed/9292394
Hawkins, R. B., Raymond, S. L., Stortz, J. A., Horiguchi, H., Brakenridge, S. C., Gardner, A., Efron, P. A., Bihorac, A., Segal, M., Moore, F. A., & Moldawer, L. L. (2018).
Chronic Critical Illness and the Persistent Inflammation, Immunosuppression, and Catabolism Syndrome. Frontiers in Immunology, 9, 1511. https://doi.org/10.3389/fimmu.2018.01511
Hoffman, R. W., & Greidinger, E. L. (2000). Mixed connective tissue disease. Current Opinion in Rheumatology, 12(5), 386-390. https://doi.org/10.1097/00002281-200009000-00006
Holliger, P., & Hudson, P. J. (2005). Engineered antibody fragments and the rise of single domains. Nature Biotechnology, 23(9), 1126-1136. https://doi.org/10.1038/nbt1142
Horvath, G. L., Schrum, J. E., De Nardo, C. M., & Latz, E. (2011). Intracellular sensing of microbes and danger signals by the inflammasomes. Immunological Reviews, 243(1), 119-135. https://doi.org/10.1111/j. 1600-065X.2011.01050.x
Hu, K.-Q. (2002). Occult hepatitis B virus infection and its clinical implications. Journal of Viral Hepatitis, 9(4), 243-257. https://doi.org/10.1046/j. 1365-2893.2002.00344.x
Hung, I. F. N. (2020). Treatment of coronavirus disease 2019. Current Opinion in HIV and AIDS, 15(6), 336-340. https://doi.org/10.1097/COH.0000000000000652
Jones, P. T., Dear, P. H., Foote, J., Neuberger, M. S., & Winter, G. (1986). Replacing the complementarity-determining regions in a human antibody with those from a mouse. Nature, 321(6069), 522-525. https://doi.org/10.1038/321522a0
Kabat E. A., Wu T. T., Perry H. M., Foeller C., G. K. S. (1991). Sequences of Proteins of Immunological Interest (5th ed.). U.S. Department of Health and Human Services, National Institutes for Health.
Kamar, N., Izopet, J., Pavio, N., Aggarwal, R., Labrique, A., Wedemeyer, H., & Dalton, H. R. (2017). Hepatitis E virus infection. Nature Reviews. Disease Primers, 3, 17086. https://doi.org/10.1038/nrdp.2017.86
Kapingidza, A. B., Kowal, K., & Chruszcz, M. (2020). Antigen-Antibody Complexes. Sub-Cellular Biochemistry, 94, 465-497. https://doi.org/10.1007/978-3-030-41769-7_19
Kittiworakarn, J., Lecoq, A., Moine, G., Thai, R., Lajeunesse, E., Drevet, P., Vidaud, C., Ménez, A., & Leonetti, M. (2006). HIV-1 Tat raises an adjuvant-free humoral immune response controlled by its core region and its ability to form cysteine-mediated oligomers. The Journal of Biological Chemistry, 281(6), 3105-3115. https://doi.org/10.1074/jbc.M509899200
Köhler, G., & Milstein, C. (1975). Continuous cultures of fused cells secreting antibody of predefined specificity. Nature, 256(5517), 495-497. https://doi.org/10.1038/256495a0
Lecoq, A., Moine, G., Bellanger, L., Drevet, P., Thai, R., Lajeunesse, E., Ménez, A., & Léonetti, M. (2008). Increasing the humoral immunogenic properties of the HIV-1 Tat protein using a ligand-stabilizing strategy. Vaccine, 26(21), 2615-2626. https://doi.org/10.1016/j.vaccine.2008.02.057
Leung, D. T. M., Tam, F. C. H., Ma, C. H., Chan, P. K. S., Cheung, J. L. K., Niu, H., Tam, J. S. L., & Lim, P. L. (2004). Antibody response of patients with severe acute respiratory syndrome (SARS) targets the viral nucleocapsid. The Journal of Infectious Diseases, 190(2), 379-386. https://doi.org/10.1086/422040
Migliorini, P., Baldini, C., Rocchi, V., & Bombardieri, S. (2005). Anti-Sm and anti-RNP antibodies. Autoimmunity, 38(1), 47-54. https://doi.org/10.1080/08916930400022715
Mimoto, F., Katada, H., Kadono, S., Igawa, T., Kuramochi, T., Muraoka, M., Wada, Y., Haraya, K., Miyazaki, T., & Hattori, K. (2013). Engineered antibody Fc variant with selectively enhanced FcγRIIb binding over both FcγRIIa(R131) and FcγRIIa(H131). Protein Engineering, Design & Selection: PEDS, 26(10), 589-598. https://doi.org/10.1093/protein/gzt022
Ni, L., Ye, F., Cheng, M.-L., Feng, Y., Deng, Y.-Q., Zhao, H., Wei, P., Ge, J., Gou, M., Li, X., Sun, L., Cao, T., Wang, P., Zhou, C., Zhang, R., Liang, P., Guo, H., Wang, X., Qin, C.-F., . . . Dong, C. (2020). Detection of SARS-COV-2-Specific Humoral and Cellular Immunity in COVID-19 Convalescent Individuals. Immunity, 52(6), 971-977.e3. https://doi.org/10.1016/j.immuni.2020.04.023
OhAinle, M., Balmaseda, A., Macalalad, A. R., Tellez, Y., Zody, M. C., Saborío, S., Nuñez, A., Lennon, N. J., Birren, B. W., Gordon, A., Henn, M. R., & Harris, E. (2011). Dynamics of dengue disease severity determined by the interplay between viral genetics and serotype-specific immunity. Science Translational Medicine, 3(114), 114ra128. https://doi.org/10.1126/scitranslmed. 3003084
Pisetsky, D. S., & Lipsky, P. E. (2020). New insights into the role of antinuclear antibodies in systemic lupus erythematosus. Nature Reviews. Rheumatology, 16(10), 565-579. https://doi.org/10.1038/s41584-020-0480-7
Riechmann, L., Clark, M., Waldmann, H., & Winter, G. (1988). Reshaping human antibodies for therapy. Nature, 332(6162), 323-327. https://doi.org/10.1038/332323a0
Rother, R. P., Rollins, S. A., Mojcik, C. F., Brodsky, R. A., & Bell, L. (2007). Discovery and development of the complement inhibitor eculizumab for the treatment of paroxysmal nocturnal hemoglobinuria. Nature Biotechnology, 25(11), 1256-1264. https://doi.org/10.1038/nbt1344
Rumbaugh, J. A., Bachani, M., Li, W., Butler, T. R., Smith, K. J., Bianchet, M. A., Wang, T., Prendergast, M. A., Sacktor, N., & Nath, A. (2013). HIV immune complexes prevent excitotoxicity by interaction with NMDA receptors. Neurobiology of Disease, 49, 169-176. https://doi.org/10.1016/j.nbd.2012.08.013
Schulte-Pelkum, J., Fritzler, M., & Mahler, M. (2009). Latest update on the Ro/SS-A autoantibody system. Autoimmunity Reviews, 8(7), 632-637. https://doi.org/10.1016/j.autrev.2009.02.010
Sospedra, M., & Martin, R. (2016). Immunology of Multiple Sclerosis. Seminars in Neurology, 36(2), 115-127. https://doi.org/10.1055/s-0036-1579739
Stadlbauer, D., Amanat, F., Chromikova, V., Jiang, K., Strohmeier, S., Arunkumar, G. A., Tan, J., Bhavsar, D., Capuano, C., Kirkpatrick, E., Meade, P., Brito, R. N., Teo, C., McMahon, M., Simon, V., & Krammer, F. (2020). SARS-COV-2 Seroconversion in Humans: A Detailed Protocol for a Serological Assay, Antigen Production, and Test Setup. Current Protocols in Microbiology, 57(1), e100. https://doi.org/10.1002/cpmc. 100
Strohl, W. R., & Strohl, L. M. B. T.-T. A. E. (Eds.). (2012). 11—IgG glycans and glyco-engineering. In Woodhead Publishing Series in Biomedicine (pp. 251-595). Woodhead Publishing. https://doi.org/https://doi.org/10.1533/9781908818096.251
Tarisse, C. F., Goulard-Huet, C., Nia, Y., Devilliers, K., Marcé, D., Dambrune, C., Lefebvre, D., Hennekinne, J.-A., & Simon, S. (2021). Highly Sensitive and Specific Detection of Staphylococcal Enterotoxins SEA, SEG, SEH, and SEI by Immunoassay. Toxins, 13(2). https://doi.org/10.3390/toxins13020130
Temming, A. R., Bentlage, A. E. H., de Taeye, S. W., Bosman, G. P., Lissenberg-Thunnissen, S. N., Derksen, N. I. L., Brasser, G., Mok, J. Y., van Esch, W. J. E., Howie, H. L., Zimring, J. C., & Vidarsson, G. (2020). Cross-reactivity of mouse IgG subclasses to human Fc gamma receptors: Antibody deglycosylation only eliminates IgG2b binding. Molecular Immunology, 127, 79-86. https://doi.org/10.1016/j.molimm.2020.08.015
Tillett, R. L., Sevinsky, J. R., Hartley, P. D., Kerwin, H., Crawford, N., Gorzalski, A., Laverdure, C., Verma, S. C., Rossetto, C. C., Jackson, D., Farrell, M. J., Van Hooser, S., & Pandori, M. (2021). Genomic evidence for reinfection with SARS-COV-2: a case study. The Lancet. Infectious Diseases, 21(1), 52-58. https://doi.org/10.1016/S1473-3099(20) 30764-7
Tisoncik, J. R., Korth, M. J., Simmons, C. P., Farrar, J., Martin, T. R., & Katze, M. G. (2012). Into the eye of the cytokine storm. Microbiology and Molecular Biology Reviews: MMBR, 76(1), 16-32. https://doi.org/10.1128/MMBR.05015-11
To, C. H., & Petri, M. (2005). Is antibody clustering predictive of clinical subsets and damage in systemic lupus erythematosus? Arthritis and Rheumatism, 52(12), 4003-4010. https://doi.org/10.1002/art.21414
Torres, D. de A., Ribeiro, L. do C. B., Riello, A. P. de F. L., Horovitz, D. D. G., Pinto, L. F. R., & Croda, J. (2020). Reinfection of COVID-19 after 3 months with a distinct and more aggressive clinical presentation: Case report. Journal of Medical Virology, jmv.26637. https://doi.org/10.1002/jmv.26637
Vafa, O., Gilliland, G. L., Brezski, R. J., Strake, B., Wilkinson, T., Lacy, E. R., Scallon, B., Teplyakov, A., Malia, T. J., & Strohl, W. R. (2014). An engineered Fc variant of an IgG eliminates all immune effector functions via structural perturbations. Methods (San Diego, Calif.), 65(1), 114-126. https://doi.org/10.1016/j.ymeth.2013.06.035
Verhoeyen, M., Milstein, C., & Winter, G. (1988). Reshaping human antibodies: grafting an antilysozyme activity. Science (New York, N.Y.), 239(4847), 1534-1536. https://doi.org/10.1126/science.2451287
Verhoeyen, M., & Riechmann, L. (1988). Engineering of antibodies. BioEssays: News and Reviews in Molecular, Cellular and Developmental Biology, 8(2), 74-78. https://doi.org/10.1002/bies. 950080207
Wang, X., Mathieu, M., & Brezski, R. J. (2018). IgG Fc engineering to modulate antibody effector functions. Protein & Cell, 9(1), 63-73. https://doi.org/10.1007/s13238-017-0473-8
Zohar, T., Loos, C., Fischinger, S., Atyeo, C., Wang, C., Slein, M. D., Burke, J., Yu, J., Feldman, J., Hauser, B. M., Caradonna, T., Schmidt, A. G., Cai, Y., Streeck, H., Ryan, E.
T., Barouch, D. H., Charles, R. C., Lauffenburger, D. A., & Alter, G. (2020). Compromised Humoral Functional Evolution Tracks with SARS-COV-2 Mortality. Cell, 183(6), 1508-1519.e12. https://doi.org/10.1016/j.cell.2020.10.052

1-15. (canceled)

16. A method of treating or preventing inflammation in a subject in need thereof, comprising administering an effective amount of an antibody specific for a protein antigen capable of binding to nucleic acids, or an antigen-binding fragment or derivative thereof, wherein the antibody, fragment or derivative has a reduced FcγRIIA-receptor binding capacity and/or an increased FcγRIIB-receptor binding capacity

17. The method according to claim 16, wherein the inflammation is due to:
a) an infection; or
b) an inflammatory or autoimmune disease.

18. The method according to claim 17, wherein:
a) the infection is a viral infection; or
b) the inflammatory or autoimmune disease is selected from rheumatoid arthritis, Kawasaki disease, systemic lupus erythematosus, systemic sclerosis and primary Sjögren's syndrome.

19. The method according to claim 18, wherein the viral infection is selected from COVID-19, influenza, AIDS, hepatitis B, hepatitis E, and dengue.

20. The method according to claim 16, wherein the antibody, fragment or derivative thereof is a whole antibody of IgG4 isotype or IgG2-IgG4 cross isotype.

21. The method according to claim 16, wherein the antibody, fragment or derivative has:
a) a reduced capacity for binding to FcγRIIA and FcγRIIIA receptors, or
b) a reduced capacity for binding to all FcγRs.

22. The method according to claim 21, wherein the antibody, fragment or derivative is a fragment or a derivative without Fc domain.

23. The method according to claim 22, wherein the fragment or a derivative without Fc domain is selected from F(ab′)2, F(ab′), Fab, Fab′, Fv, VhH, V-NAR, ScFv, Bis-scFv, Fab₂, Fab₃, diabodies, triabodies and tetrabodies.

24. The method according to claim 21, wherein the antibody, fragment or derivative is a whole antibody of IgG isotype which has one or more mutations in the Fc domain significantly reducing its binding to FcγRIIA, to FcγRIIA and FcγRIIIA or to all FcγRs.

25. The method according to claim 24, wherein the antibody is of IgG1 isotype and its Fc domain comprises a combination of the 4 mutations E233P, F234V, L235A, and D265A.

26. The method according to claim 21, wherein the antibody, fragment or derivative is a whole antibody of IgG isotype which is not glycosylated in the Fc domain.

27. The method according to claim 16, wherein the antibody, fragment or derivative is a whole antibody of IgG isotype which has one or more mutations in the Fc domain significantly increasing its binding to FcγRIIb.

28. The method according to claim 16, wherein the protein antigen capable of binding to nucleic acids has one of the following features:
(a) it is capable of binding to nucleic acids alone, without being complexed to another molecule;
(b) it is capable of binding to nucleic acids without specificity for a given nucleic sequence;
(c) It is capable of binding to nucleic acids more efficiently at a pH comprised between 7.0 and 7.5 than at acidic pH;
(d) it comprises one or more positively-charged accessible region;
(e) it is also capable of binding to membrane heparan sulfate proteoglycans, preferably alone, without being complexed to another molecule; and
(f) any combination of features (a) to (e).

29. The method according to claim 16, wherein the protein antigen capable of binding to nucleic acids is selected from viral proteins capable of binding to the viral genome.

30. The method according to claim 29, wherein the viral proteins capable of binding to the viral genome are selected from:
viral capsid proteins, and
the transcriptional transactivator (Tat) and reverse transcriptase (RT, p66/p51) of HIV-1.

31. The method according to claim 29, wherein the viral capsid proteins are selected from capsid proteins of HIV-1, SARS-COV-2, influenza virus, HBV, HCV, HEV, and HPV.

32. The method according to claim 16, wherein the protein antigen capable of binding to nucleic acids is selected from self proteins of the subject to be treated.

33. The method according to claim 32, wherein the self proteins of the subject to be treated are selected from ribonucleoproteins and deoxyribonucleoproteins of the subject to be treated.

34. The method according to claim 33, wherein:
a) the ribonucleoprotein is selected from the proteins RibP, snRNP, Ro60, Ro52, and lupus antigen La, or
b) the deoxyribonucleoprotein is a histone.
